SEQUENCE LISTING (11 sequences)

SEQ ID NO: 1 - DNA, 858 bp, Porcine
  CDS (1)..(858)
atgacagaagaggaggtgcggtttgaagggatattttttcctaaacagattctctcgcctgaaatgctgcaagaggtgagggaggaatttacatttaaagaggaagatgttttaatactgacttttcccaaatcaggaaccaactggatgattgaaatcatctgcctgattctctccaagggggataccaagtggatccagtctgtacccaactgggatcgctcgccctggctagagagtatctctgggtatgaaaatctcaagaacaaggaaggcccacgcctcatcagttcccacctccccatccaactcttccccaaggctttcttcaagtccaaggccaagatgatttacatcatcagaaatcccagagatattattgtgtctggttattttttctggaagtctacaaatttagttaagagaccagagtcactggaacaatattttgaatggttcatccaaggaaatgtgccctatggatcatggtttgaccacattcgtggctggttgcccatgcgagacaaggagaacgtcctgatactcagctatgaagagctgaaacgggacacgagaagcgccgtagagaaaatctgccagttcctggggaagaagctagaacccgaggaactgagctcggtcgtcgaaaacagctccttccaggtcatgaaagaaaacaacatgtccaatttttccctcctgaagggtttgcatcttggggacactggatgtcttttgagaaaaggcactcctggggactggaaaaactacttcacggtggcccaagccgaagcctttgataaactattccaggagaagatggcagatcttcctcaagagctgttcccatggcaataa

SEQ ID NO: 2 - protein, 285 aa, Porcine
MTEEEVRFEGIFFPKQILSPEMLQEVREEFTFKEEDVLILTFPKSGTNWMIEIICLILSKGDTKWIQSVPNWDRSPWLESISGYENLKNKEGPRLISSHLPIQLFPKAFFKSKAKMIYIIRNPRDIIVSGYFFWKSTNLVKRPESLEQYFEWFIQGNVPYGSWFDHIRGWLPMRDKENVLILSYEELKRDTRSAVEKICQFLGKKLEPEELSSVVENSSFQVMKENNMSNFSLLKGLHLGDTGCLLRKGTPGDWKNYFTVAQAEAFDKLFQEKMADLPQELFPWQ

SEQ ID NO: 3 - DNA, 1036 bp, porcine
acgcggggaatgacagctgaaacgctcccacgaggcgcaaagaactcagcatcatgacagaagaggaggtgcggtttgaagggatattttttcctaaacagattctctcgcctgaaatgctgcaagaggtgagggaggaatttacatttaaagaggaagatgttttaatactgacttttcccaaatcaggaaccaactggatgattgaaatcatctgcctgattctctccaagggggataccaagtggatccagtctgtacccaactgggatcgctcgccctggctagagagtatctctgggtatgaaaatctcaagaacaaggaaggcccacgcctcatcagttcccacctccccatccaactcttccccaaggctttcttcaagtccaaggccaagatgatttacatcatcagaaatcccagagatattattgtgtctggttattttttctggaagtctacaaatttagttaagagaccagagtcactggaacaatattttgaatggttcatccaaggaaatgtgccctatggatcatggtttgaccacattcgtggctggttgcccatgcgagacaaggagaacgtcctgatactcagctatgaagagctgaaacgggacacgagaagcgccgtagagaaaatctgccagttcctggggaagaagctagaacccgaggaactgagctcggtcgtcgaaaacagctccttccaggtcatgaaagaaaacaacatgtccaatttttccctcctgaagggtttgcatcttggggacactggatgtcttttgagaaaaggcactcctggggactggaaaaactacttcacggtggcccaagccgaagcctttgataaactattccaggagaagatggcagatcttcctcaagagctgttcccatggcaataatgttcacaaacttcccaatcctctatggatattgatattcgctcacctgtccttgcacatgtgaatgatgggggctaattacacaaaaacctcttgtttcatccttgaaaaaaaaaaaaaaaaaa

SEQ ID NO: 4 - DNA, 18 bp, Porcine
cacgaggcgcaaagaact

SEQ ID NO: 5 - DNA, 20 bp, Porcine
catgtgcaaggacaggtgag

SEQ ID NO: 6 - DNA, 20 bp, Porcine
ccatgcgagacaaggagaac

SEQ ID NO: 7 - DNA, 20 bp, Porcine
catgacctggaaggagctgt

SEQ ID NO: 8 - DNA, 20 bp, Porcine
ctttgctgacctgctggatt

SEQ ID NO: 9 - DNA, 20 bp, Porcine
cttgaccaaggaaagcaagg

SEQ ID NO: 10 - protein, 285 aa, Porcine; identical to SEQ ID NO: 2

SEQ ID NO: 11 - protein, 285 aa, Homo Sapiens [sequence omitted]

(19) United States
(12) Patent Application Publication
(10) Pub. No.: US 2006/0024708 A1
(43) Pub. Date: Feb. 2, 2006

(54) Porcine sulfotransferase 2A1 polynucleotide sequence, protein, and methods of use for same

(75) Inventors: E. James Squires, Guelph (CA); Philip Sinclair, Guelph (CA)

Correspondence Address: MCKEE, VOORHEES & SEASE, P.L.C., 801 GRAND AVENUE, SUITE 3200, DES MOINES, IA, 50309-2721, US

(73) Assignee: UNIVERSITY OF GUELPH, Guelph (CA)

(21) Appl. No.: 11/152,635

(22) Filed: Jun. 14, 2005

Related U.S. Application Data
(60) Provisional application No. 60/580,540, filed on Jun. 17, 2004.

(51) Int. Cl. C12Q 1/68; C07H 21/04; C12P 21/06; C12N 9/10

(52) U.S. Cl. 435/6; 435/69.1; 435/193; 435/320.1; 435/325; 536/23.2

(57) ABSTRACT

The invention pertains to the identification and isolation of a gene involved in the boar taint phenotype. A porcine form of a gene, designated SULT2A1, is identified. The invention additionally provides methods of inhibiting or reducing boar taint using the novel SULT2A1 gene or gene product. Methods for reducing or inhibiting boar taint and methods for screening compounds to modulate SULT2A1 are also disclosed.

### CROSS-REFERENCE TO RELATED APPLICATIONS

This application claims priority under 35 U.S.C. § 119 of a provisional application Ser. No. 60/580,540 filed Jun. 17, 2004, which application is hereby incorporated by reference in its entirety.

### BACKGROUND OF THE INVENTION

The present invention provides a nucleic acid sequence that encodes a protein molecule that has been identified as a member of the enzyme family of proteins known as sulfotransferases. The present invention also provides isolated polypeptide sequences encoded by the nucleic acid sequence. The invention also provides polymorphic nucleotide sequences from a porcine hydroxysteroid sulfotransferase SULT2A1 gene. Also provided by the present invention are methods and compositions for regulating boar taint by decreasing the levels of 5α-androstenone that accumulates in adipose tissue.

Males pigs used for pork production are castrated early in life to prevent boar taint or sex taint in the meat. However, castration also removes sources of natural anabolic androgens that stimulate lean growth. As a result, uncastrated (intact) males have improved feed efficiency and greater lean yield of the carcass compared to barrows. Therefore, the prevention of boar taint without castration is very desirable.

The development of genetic markers for pigs that are low in boar taint would allow the selection of lines of pigs that are free boar taint. Candidate genes which function and code for proteins that affect this trait are also desirable.

Rearing intact male pigs for pork production can be advantageous due to their superior production characteristics (Andresen, 1976). Specifically, intact males have an improved feed conversion efficiency (Fortin et al., 1983; Squires et al., 1993) as well as an increase in lean meat production compared to either castrates or gilts (Kempster and Lowe, 1993; Sather et al., 1991). In addition, the fatty tissue in intact males has a higher overall percentage of unsaturated fats compared to castrates and gilts (Babol and Squires, 1995; Squires et al., 1993). These differences combine to produce a healthier product for the consumer, as well as a potential financial gain for the producer (deLange and Squires, 1995; Walstra and Vermeer, 1993). However, a small percentage of meat from intact males contains what is known as ‘boar taint’ (Gower, 1972). Boar taint, first described chemically by Patterson (1968), refers to the unpleasant ‘urine/perspiration-like’ odor that is associated with boar fat when it is heated during cooking. Consequently, all male pigs intended for pork production in North America are castrated shortly after birth to avoid the adverse effects of boar taint.

Castration soon after birth effectively prevents the development of boar taint; however, it also removes the anabolic effects of testicular steroids, which are responsible for the production advantages of boars. In addition to the lower production efficiency that is associated with barrows, castration raises substantial concerns for animal welfare, with potential increases in morbidity. It has therefore become increasingly important to find methods other than castration to prevent boar taint. The ability to identify animals that are genetically prone to developing a tainted carcass would allow for selection and the use of non-tainted boars for pork production.

Boar taint can be attributed to high concentrations of the testicular steroid 5α-androst-16-en-3-one (5α-androstenone) (Gower and Patterson, 1970; Patterson, 1968) and 3-methylindole, a naturally occurring microbial metabolite of tryptophan (Yokoyama and Carlson, 1979), that accumulate in adipose tissue. Some studies have indicated that androstenone is the main contributor (Babol et al., 1996; Bonneau et al., 1982; Malmfors and Andresen, 1975), whereas others have suggested that 3-methylindole has a larger impact (Andresen et al., 1993; Bejerholm and Barton-Gade, 1993). 3-Methylindole is present in all sexes, suggesting that the high levels that accumulate in the adipose tissue of intact males are due to a decrease in metabolic clearance which is influenced by the anabolic status of the male.

Approximately five to 15 percent of market weight boars in North America have fat concentrations of 5α-androstenone that are high enough to be considered offensive by consumers (Malmfors and Lundstrom, 1983; Squires and Lou, 1995). The cut-off level of 5α-androstenone in fat has not been precisely determined; however, it ranges from 0.5 to 1.0 μg/g (Malmfors and Lundstrom, 1983). This range in the threshold value is the result of a number of factors, the most significant of which is the individual differences in the sensory perception of boar taint by consumers (Griffiths and Patterson, 1970; Wysocki and Beauchamp, 1984). Despite these differences in sensory perception, large inter-individual differences in the level of 5α-androstenone accumulation are present within and between breeds. Contributing factors such as breed, stage of maturity, and genotype can influence the levels of 5α-androstenone in fat (Bonneau, 1987; Sellier and Bonneau, 1988). Distinct breed differences have been identified in a number of studies. Between five to eight percent of purebred Hampshire, Yorkshire and Landrace boars have high concentrations of 5α-androstenone in fat, whereas 50 percent of Duroc intact males have high concentrations (Squires et al., 1992; Xue et al., 1996).

Increases in 5α-androstenone concentrations in fat are generally observed between five and six months of age as the animal approaches market weight and physiological maturity (Andresen, 1976; Claus et al., 1994; Sinclair et al., 2001a). The increased level of 5α-androstenone in fat at puberty coincides with the increase in testicular 16-androstene steroidogenesis at this time (Schwarzenberger et al., 1993; Sinclair et al., 2001b). Low levels of 5α-androstenone measured in some market weight boars may be a consequence of immaturity and not due to a genetic predisposition to decreased boar taint. Despite the influence of maturity at market weight, genetic selection for animals with low boar taint may be effective (Willeke et al., 1987; Willeke and Pirchner, 1989) due to the relatively high heritability (0.56) of fat 5α-androstenone (Bonneau and Sellier, 1986; Sellier and Bonneau, 1988). Unfortunately, selection for low 5α-androstenone concentrations coincides with selection for low androgen production and poor reproductive performance (Willeke et al., 1987). It is therefore desirable to identify animals that have a decreased genetic capacity to accumulate 5α-androstenone in fat while maintaining the normal levels of testicular steroids that are characteristic of intact males. Therefore, the development of genetic markers for pigs that are low in boar taint would allow the selection of lines of pigs that are free of taint.

Past research has generally focused on identifying animals that have a decreased genetic capacity to produce the 16-androstene steroids (Babol et al., 1999; Davis and Squires, 1999; Louveau et al., 1991); however, the amount of 5α-androstenone that is available to accumulate in fat is not necessarily a direct result of testicular synthesis. Metabolism and clearance from the body, and the genetic capacity to do so, will greatly affect how much 5α-androstenone is present within the circulation to accumulate in fat. Thus the level of 5α-androstenone accumulation in fat is a result of the balance between testicular steroidogenesis and metabolic clearance of the 16-androstene steroids.

Sulfoconjugation is a major conjugation reaction that is involved in the metabolism of a variety of endogenous and xenobiotic compounds. The sulfation of steroids involves the transfer of a sulfate (SO₃—) from an activated donor molecule to a hydroxyl acceptor site. The donor molecule for the transfer of the sulfate moiety has been identified as 3′-phosphoadenosine 5′phosphosulfate (PAPS) (Robbins, 1956). This donor molecule serves as the supplier of sulfate for a multitude of biological processes. PAPS is synthesized from ATP and SO₄²⁻, and this reaction is catalyzed by the PAPS synthetase enzyme, which is localized in the cytosol (Lyle et al., 1994; Xu et al., 2001). Sources of inorganic sulfate required for synthesis of PAPS originate from the diet and amino acid catabolism (Falany, 1997).

The transfer of the sulfate moiety from PAPS to an acceptor site on a steroid is catalyzed by the sulfotransferase family of enzymes (Roy, 1970). These enzymes are mainly located in the cytosolic compartment and are capable of conjugating steroids as well as a wide range of drugs and xenobiotics. Sulfoconjugation brings about a striking change in the physiochemical properties of steroids. With the addition of the highly charged sulfate group the polarity of the steroid increases, causing an increase in water solubility (Bongiovanni and Cohn, 1970; Jakoby et al., 1980).

Because of the potentially significant role of sulfoconjugation of the 16-androstene steroids in the development of boar taint, it is important to understand the molecular basis for individual variation in the expression and function of the SULT2A1 gene in market weight boars. We have shown that hydroxysteroid sulfotransferase (SULT2A1) is a key enzyme in the testicular and hepatic metabolism of 5α-androstenone in pig; thus it would be desirable to modulate the expression or activity of SULT2A1 to reduce boar taint. Moreover, investigation into how porcine SULT2A1 genetic variation translates into interindividual differences in 5α-androstenone accumulation in fat is of great importance in warranting the development of genetic markers for pigs that will efficiently metabolize and clear 16-androstene steroids, so they will not be tainted. It is therefore advantageous to identify animals having polymorphisms in this candidate gene for identifying low boar taint pigs.

### BRIEF SUMMARY OF THE INVENTION

The invention provides the isolated nucleotide sequence of porcine SULT2A1 or fragments thereof and nucleic acid sequences that hybridize to SULT2A1, as well as methods of using the SULT2A1 gene, for example, to reduce boar taint, and/or to enhance the expression of a SULT2A1 gene to decrease the amount of 5α-androstenone that is available to accumulate in fat. This sequence is also polymorphic and thus may be used for identifying genetic markers of boar taint based upon different forms of SULT2A1. The development of genetic markers for pigs that are low in boar taint would allow the selection of pigs or development of lines of pigs that are low in boar taint.

In a still further embodiment, the invention provides a polynucleotide that encodes a SULT2A1 variant (polymorphism or mutation). In one embodiment, a polymorphic SULT2A1 sequence comprises the following polymorphisms: A cytosine (C) to thymine (T) substitution at nucleotide 219 of SEQ ID NO:1.

The invention further provides a vector that directs the expression of SULT2A1, and a host cell transfected or transduced with this vector. The recombinant host cells expressing the polypeptide described herein have a variety of uses. The cells are useful for producing an enzyme protein or peptide that can be further purified to produce desired amounts of enzyme protein or fragments. Thus, host cells containing expression vectors are useful for peptide production.

Host cells are also useful for conducting cell-based assays involving the enzyme protein or enzyme protein fragments, such as those described above as well as other formats known in the art. Thus, a recombinant host cell expressing a native enzyme protein is useful for assaying compounds that stimulate SULT2A1 enzyme protein function. Cell-based, in vitro assays of cellular function contribute significantly to a better understanding 16-androstene steroid metabolism and its impact on the development of boar taint, or the effect of a compound on SULT2A1 activity.

Host cells are also useful for identifying enzyme protein mutants in which these functions are affected. If the mutants naturally occur and give rise to a condition such as boar taint, host cells containing the mutations are useful to assay compounds that have a desired effect on the mutant enzyme protein (for example, stimulating function) which may not be indicated by their effect on the native enzyme protein.

In another aspect, the invention relates to an isolated polypeptide encoded by the SULT2A1 gene or a fragment thereof, and antibodies generated against the SULT2A1 polypeptide, peptides, or portions thereof, which can be used to detect, and/or reduce boar taint.

Yet another aspect of the invention is a method of reducing boar taint by enhancing the activity of SULT2A1 in a pig. The activity of SULT2A1 can be enhanced by administering (a) a substance that increases the activity of a sulfotransferase enzyme; or (b) a substance that induces or increases the expression of the SULT2A1 gene. In another embodiment, the activity of the SULT2A1 enzyme can also be enhanced by administering a nucleic acid sequence encoding a SULT2A1 enzyme into a pig, either ex vivo or in vivo.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used herein, the term “SULT2A1 gene” is intended to generically refer to both the wild-type and variant forms of the sequence, unless specifically denoted otherwise. The nucleotide sequence of SULT2A1 is depicted in FIG. 2.

As used herein, the term “SULT2A1 activity” is intended to broadly refer to sulfation of hydroxysteroids.

The term “polymorphism”, as used herein, refers to a difference in the nucleotide or amino acid sequence of a given region as compared to a nucleotide or amino acid sequence in a homologous region of another animal, in particular, a difference in the nucleotide or amino acid sequence of a given region which differs between animals of the same species. A polymorphism is generally defined in relation to a reference or wild-type sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism, for example http://darwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

As used herein, the term “polymorphic SULT2A1 nucleic acid” refers to a polynucleotide derived from a SULT2A1 gene comprising one or more polymorphisms when compared to a reference SULT2A1 polynucleotide sequence. A polymorphism in a polymorphic SULT2A1 nucleic acid may be one that is associated with a condition relating to SULT2A1 activity.

The terms “polynucleotide” and “nucleic acid molecule” are used interchangeably herein to refer to wild type or polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The α (alpha)-anomer of deoxyribose may be used, where the base is inverted with respect to the natural β (beta)-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The term “polypeptide” refers to a polymer of amino without regard to the length of the polymer; thus, peptides, oligopeptides, and proteins are included within the definition of polypeptide. This term also does not specify or exclude post-translation modifications of polypeptides. For example, polypeptides that include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

Hybridization reactions can be performed under conditions of different “stringency”. Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1 ×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. Examples of stringent conditions are hybridization and washing at 50° C. or higher and in 0.1×SSC (9 mM NaCl/0.9 mM sodium citrate). “Tₘ” is the temperature in degrees Celsius at which 50% of a polynucleotide duplex made of complementary strands hydrogen bonded in anti-parallel direction by Watson-Crick base pairing dissociates into single strands under conditions of the experiment. Tm may be predicted according to a standard formula, such as:
Tₘ=81.5+16.6 log[X⁺]+0.41 (% G/C)−0.61 (% F)−600/L
where [X⁺] is the cation concentration (usually sodium ion, Na⁺) in mol/L; (% G/C) is the number of G and C residues as a percentage of total residues in the duplex; (% F) is the percent formamide in solution (wt/vol); and L is the number of nucleotides in each strand of the duplex.

Stringent conditions for both DNA/DNA and DNA/RNA hybridization are as described by Sambrook et al. Molecular Cloning, A Laboratory Manual, 2nd Ed., Cold Spring Harbor Laboratory Press, Cold Spring Harbor, N.Y., 1989, herein incorporated by reference. By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll. 0.02% BSA, and 500 μg/ml denatured salmon sperm DNA. Filters are hybridized for 48 h at 65° C. the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989; and Ausubel et al., 1989, are incorporated herein in their entirety.

These hybridization conditions are suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in the book of Hames and Higgins (1985) or in Sambrook et al. (1989).

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. In this case the reference sequence is the porcine wild-type SULT2A1 gene. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that the C to T polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene (e.g. the gene discussed herein), which have previously been shown to be associated with a particular trait, e.g., boar taint. Thus, in the present situation, taking one or both of the genes, it would be possible, at least in the short term, to select for animals likely to produce desired traits, or alternatively against animals likely to produce less desirable traits indirectly, by selecting for certain alleles of an associated marker through the selection of specific alleles of alternative chromosome markers.

As used herein, an “isolated” nucleic acid molecule is one that is separated from other nucleic acid present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5 kb, 4 kb, 3 kb, 2 kb, or 1 kb or less, particularly contiguous peptide encoding sequences and peptide encoding sequences within the same gene but separated by introns in the genomic sequence. The important point is that the nucleic acid is isolated from remote and unimportant flanking sequences such that it can be subjected to the specific manipulations described herein such as recombinant expression, preparation of probes and primers, and other uses specific to the nucleic acid sequences.

Moreover, an “isolated” nucleic acid, such as a transcript/cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated. For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

The term “vector” refers to a vehicle, preferably a nucleic acid molecule, which can transport the nucleic acid molecules.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine, one exception is Micrococcus rubens, for which GTG is the methionine codon (Oshizuka, et al, J Gen'l Microbiol, 139:425-432 (1993)) can be modified to yield a functionally identical molecule. Accordingly, each silent variation of a nucleic acid, which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90%, preferably 60-90% of the native protein for it's native substrate. It is thus contemplated that various changes may be made in the peptide sequences of the disclosed compositions, or corresponding DNA sequence which encodes the peptide without appreciable loss of their biological utility or activity. A conservative substitution table providing functionally similar amino acids is provided in Table 1:

TABLE 1

| Amino Acids | Codons |
|---|---|
| Phenylalanine (Phe or F) | UUU, UUC, UUA or UUG |
| Leucine (Leu or L) | CUU, CUC, CUA or CUG |
| Isoleucine (Ile or I) | AUU, AUC or AUA |
| Methionine (Met or M) | AUG |
| Valine (Val or V) | GUU, GUC, GUA, GUG |
| Serine (Ser or S) | AGU or AGC |
| Proline (Pro or P) | CCU, CCC, CCA, CCG |
| Threonine (Thr or T) | ACU, ACC, ACA, ACG |
| Alanine (Ala or A) | GCU, GCG, GCA, GCC |
| Tryptophan (Trp) | UGG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | AGA or AGG |
| Glycine (Gly or G) | GGU or GGC or GGA or GGG |
| Termination codon | UAA, UAG or UGA |

Additionally, guidance in determining which amino acid residues can be substituted, inserted, or deleted without abolishing biological activity can be found using computer programs (e.g., LASERGENE software, DNASTAR Inc., Madison, Wis.).

The following six groups each contain amino acids that are conservative substitutions for one another:

1) Alanine (A), Serine (S), Threonine (T);

2) Aspartic acid (D), Glutamic acid (E);

3) Asparagine A), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). See also, Creighton (1984) Proteins W. H. Freeman and Company.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity such as enzyme activity or in assays such as an in vitro proliferative activity. Sites that are critical for binding partner/substrate binding can also be determined by structural analysis such as crystallization, nuclear magnetic resonance or photoaffinity labeling (Smith et al., J. Mol. Biol. 224:899-904 (1992); de Vos et al. Science 255:306-312 (1992)).

The term “antibody” as used herein refers to immunoglobulin molecules and immunologically active portions of immunoglobulin molecules, i.e., molecules that contain an antigen binding site that specifically binds an antigen.

The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

As used herein, “modulation” refers to up-regulation (i.e. activation or agonization) of nucleic acid expression.

Other features and advantages of the present invention will become apparent from the following detailed description. The accompanying figures, which are incorporated herein and which constitute a part of this specification, illustrates one embodiment of the invention and, together with the description, serve to explain the principles of the invention.

### BRIEF DESCRIPTION OF THE DRAWINGS

FIG. 1 shows the porcine SULT2A1 polynucleotide sequence (SEQ ID NO:1).

FIG. 2 shows the nucleotide sequence (SEQ ID NO:2) and deduced amino acid sequence (SEQ ID NO:3) for the porcine SULT2A1 cDNA.

FIG. 3 shows distribution of testicular (A) and hepatic (B) SULT2A1 activity against the level of 5α-androstenone present in the plasma sulfoconjugate fraction.

FIG. 4 shows the relationship between SULT2A1 activity and 5α-androstenone accumulation in fat. Values are plotted as mean±standard error (n=14). Low 5α-androstenone animals (shaded bars) with fat concentrations below 0.5 μg/g, and high 5α-androstenone animals (open bars) with fat concentrations above 0.5 μg/g. *P<0.01.

FIG. 5 shows alignment of the 285 amino acid sequence of human SULT2A1 and porcine SULT2A1, including the active site of the protein.

FIG. 6 shows porcine SULT2A1 genetic polymorphisms. (A): SSCP analysis of the SULT2A1 gene in both testicular (T) and liver (L) tissues. (B): sequence analysis of SULT2A1 for bp position 219 heterozygotes and homozygotes.

FIG. 7 shows the distribution of testicular SULT2A1 protein against enzyme activity.

FIG. 8 shows the relationship between SULT2A1 protein as determined from western immunblot analysis and 5α-androstenone accumulation in fat.

FIG. 9 shows quantitative real-time PCR of SULT2A1 expression.

### DETAILED DESCRIPTION OF THE PREFERRED EMBODIMENT

Reference will now be made in detail to the presently preferred embodiments of the invention, which together with the following examples, serve to explain the principles of the invention.

Sulfotransferase enzymes are cytosolic proteins that are involved in catalyzing the conjugation of many steroids, bile acids, and xenobiotics. Sulfotransferases utilize the donor molecule 3′-phosphoadenosine 5′phosphosulfate (PAPS) for the transfer of a sulfate radical (SO₃₋) to a hydroxyl acceptor site (Robbins, 1956). In terms of steroids, hydroxyl groups at positions 3, 21, and 17 of the steroid nucleus are the most common locations for sulfoconjugation (Strott S. A., 1996). With the addition of the sulfate group, the polarity of the steroid conjugate greatly increases, causing an increase in water solubility (Bongiovanni and Cohn, 1970; Jakoby et al., 1980). Therefore, sulfoconjugation of hydroxysteroids has been regarded as a major mechanism for their metabolism and excretion (Mulder, 1981).

Steroid sulfotransferase enzymes are located in the liver and other organs such as the adrenal glands, ovary, and testis (Gasparini et al., 1976; Hobkirk, 1985; Roberts and Lieberman, 1970). In the boar, a main organ responsible for steroid sulfate synthesis is the testes (Hobkirk et al., 1989; Raeside and Renaud, 1983). One of the major steroid sulfotransferases is hydroxysteroid sulfotransferase. Hydroxysteroid sulfotransferase has a very large substrate specificity; however, its primary substrate is dehydroepiandrosterone (DHEA) and has thus been named DHEA-sulfotransferase in the past (Comer et al., 1993; Falany et al., 1989). In recent years DHEA-sulfotransferase has been further classified to belong to the 2A family of human sulftotransferases, and is has been designated as SULT2A1 by the HUGO Nomenclature Committee. Other steroids that can serve as substrates for HST include, testosterone, androsterone and estrogens such as estrone or estradiol (Strott S. A., 1996). The sulfo conjugation of testosterone is known to occur in several tissues including the testis, adrenal, and liver (Payne, 1980; Vihko and Ruokonen, 1975). A specific testosterone sulfotransferase has not been identified; however HST will sulfate testosterone at the 17β position (Park et al., 1999). In the testes, HST is localized in the Leydig cells of multiple species, including the boar (Hobkirk et al., 1989). In humans, fetal testes express HST but are incapable of sulfating testosterone, whereas adrenal tissues expressing HST are highly capable of sulfating testosterone at this stage of development (Jaffe and Payne, 1971). Thus, steroid conjugation is likely dependant on developmental effects of the sulfotransferase enzyme and, as a consequence, metabolism may be quite different in the fetus and neonate compared to that of the adult. In terms of the estrogens, HST attaches a sulfate to the 17β-hydroxy acceptor site of estradiol, and to the phenolic acceptor site of estrone (Jakoby et al., 1980; Lyon and Jakoby, 1980; Sharp et al., 1993). However, the sulfation of estrone at this position is relatively inefficient. Estrone is more likely to be sulfated at the 3 position by estrogen sulfotransferase.

EST is specific for the 3-hydroxyl or phenolic group of estrogenic steroids (Negishi et al., 2001; Tomizuka et al., 1994), while the 17β-hydroxyl group can be sulfated by HST. Expression of EST has been found in such tissues as liver, kidney, brain, adrenal cortex (Hobkirk, 1985) and the testis (Hobkirk et al., 1989). In addition, several isoforms of EST have been identified, each of which have differing affinities for estrogens (Falany, 1997). EST is also capable of sulfoconjugating certain hydroxysteroids such as DHEA and pregnenolone, but to a lesser degree than that of HST (Falany et al., 1994; Negishi et al., 2001).

A hydroxysteroid sulfotransferase is reported here to be responsible for sulfoconjugating the 16-androstene steroids. The 16-androstene steroids are the most quantitatively abundant steroids produced by the boar testes, reaching total levels of approximately 0.6 mg/g of testicular tissue (Booth, 1975; Booth and Polge, 1976; Gower, 1972). The 16-androstene steroids are known for their involvement in boar taint (Gower, 1972; Patterson, 1968). Boar taint is partly due to the accumulation of high levels of 5α-androst-16-en-3-one (5α-androstenone) in adipose tissue, which produces an unpleasant odor upon heating or cooking of the fat. We show here that increased levels of sulfoconjugated 16-androstene steroids present in the systemic circulation are associated with a reduction in the accumulation of 5α-androstenone in adipose tissue

In humans, SULT2A1 activities have been reported to vary among individuals up to 5-fold, with individuals belonging to either low or high activity subgroups (Aksoy et al., 1993; Weinshilboum and Aksoy, 1994). These findings suggest that genetic polymorphisms may be involved in regulating enzyme activity. Single nucleotide polymorphisms (SNPs) within the human SULT2A1 gene have been observed in a number of studies (Igaz et al., 2002; lida et al., 2001; Ottemess et al., 1995a), some of which have resulted in reductions in the levels of both enzyme activity and the level of protein (Thomae et al., 2002; Wood et al., 1996).

We show here that hydroxysteroid sulfotransferase (SULT2A1) is a key enzyme in the testicular and hepatic metabolism of 5α-androstenone in pig. Testicular SULT2A1 activity was negatively correlated (r=−0.57; P<0.01) with 5α-androstenone concentrations in fat. The cDNA sequence of porcine SULT2A1 was determined and found to be highly homologous to human, mouse, and rat SULT2A1 genes. SSCP analysis was used to scan for polymorphisms within the SULT2A1 coding region from individual testes and liver samples. A mutation as disclosed herein, from a cytosine to a thymine within the coding region at bp position 219 was identified within the coding region; however, this did not affect the amino acid sequence of the enzyme. Animals with high concentrations of 5α-androstenone in fat and low SULT2A1 activity had corresponding low levels of SULT2A1 protein compared to animals with low levels of 5α-androstenone in fat. Real-time PCR analysis indicated that the expression of the SULT2A1 gene was increased 3.5 fold in animals with high levels of the protein relative to animals with low levels of the protein. These results suggest that differences in SULT2A1 expression can influence 5α-androstenone accumulation in fat. Low levels of SULT2A1 activity will result in increased levels of the unconjugated form of the steroid that can accumulate in the adipose tissue of pigs and cause boar taint.

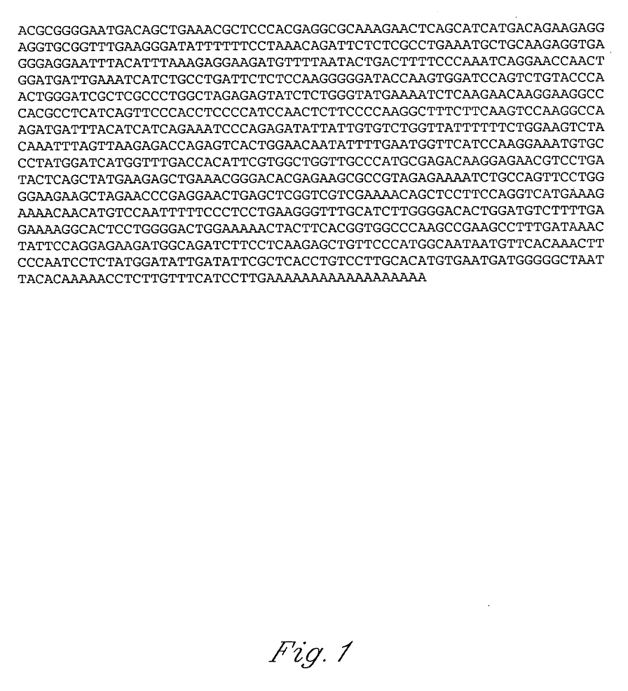

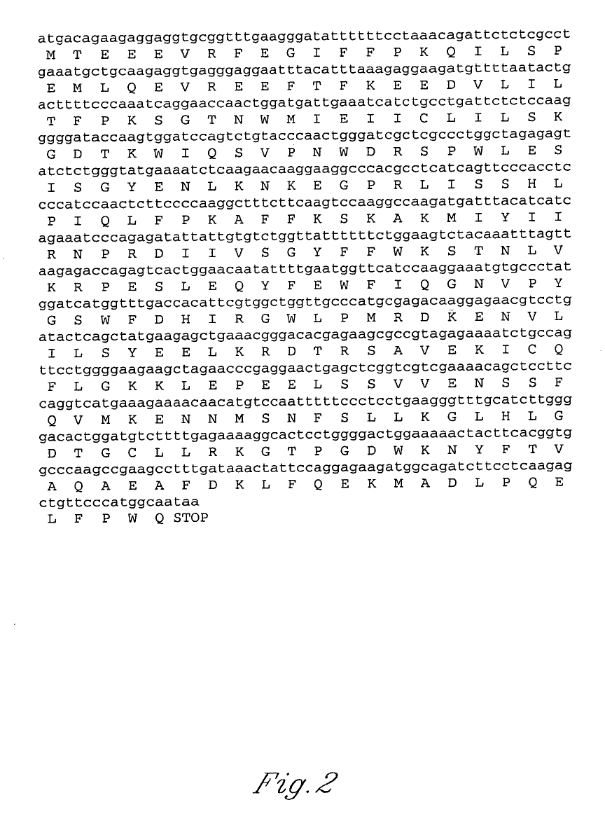

In it broadest embodiment, the present invention provides a gene that encodes a sulfotransferase enzyme which is involved in the metabolism of the 16-androstene steroids. Such nucleic acid will consist of, consist essentially of, or comprise a nucleotide sequence that encodes the enzyme peptide of the present invention, and allelic variants thereof. Accordingly, the present invention provides a novel isolated gene sequence in porcine whose difference in expression influences 5α-androstenone concentration in fat. The porcine SULT2A1 sequence is depicted in FIG. 1 (SEQ ID NO:1). In some embodiments, the present invention provides polynucleotide sequences that are capable of hybridizing to SEQ ID NO:1 under conditions of low to high stringency as long as the polynucleotide sequence capable of hybridizing encodes a protein that retains a biological activity of the naturally occurring SULT2A1. In FIG. 2 the nucleotide sequence and deduced amino acid sequence for porcine SULT2A1 cDNA is shown.

The isolated polynucleotide can encode the mature protein plus additional amino or carboxyl-terminal amino acids, or amino acids interior to the mature peptide (when the mature form has more than one peptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The isolated polynucleotide sequence includes, but is not limited to, the sequence encoding the enzyme peptide alone, the sequence encoding the mature peptide and additional coding sequences, such as a leader or secretory sequence (e.g., a pre-pro or pro-protein sequence), the sequence encoding the mature peptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the nucleic acid molecule may be fused to a marker sequence encoding, for example, a peptide that facilitates purification.

The disclosed isolated polynucleotide can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention further provides an isolated polynucleotide that encodes fragments of the peptides of the present invention as well as the polynucleotides that encodes obvious variants of the enzyme proteins of the present invention that are described above. Such polynucleotide may be naturally occurring, such as allelic variants (same locus), paralogs (different locus), and orthologs (different organism), or may be constructed by recombinant DNA methods or by chemical synthesis. Such non-naturally occurring variants may be made by mutagenesis techniques, including those applied to nucleic acid molecules, cells, or organisms. Accordingly, the variants can contain, but is not limited to, nucleotide substitutions, deletions, inversions and insertions. Variation can occur in either or both the coding and non-coding regions. The variations can produce both conservative and non-conservative amino acid substitutions.

The present invention further provides non-coding fragments of the polynucleotide sequence. Preferred non-coding fragments include, but are not limited to, promoter sequences, enhancer sequences, gene modulating sequences and gene termination sequences. Such fragments are useful in controlling heterologous gene expression and in developing screens to identify gene-modulating agents. A promoter can readily be identified as being 5′ to the ATG start site in the genomic sequence.

A fragment comprises a contiguous nucleotide sequence greater than 12 or more nucleotides. Further, a fragment could at least 30, 40, 50, 100, 250 or 500 nucleotides in length. The length of the fragment will be based on its intended use. For example, the fragment can encode epitope bearing regions of the peptide, or can be useful as DNA probes and primers. Such fragments can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of gene.

A probe/primer typically comprises substantially a purified oligonucleotide or oligonucleotide pair. The oligonucleotide typically comprises a region of nucleotide sequence that hybridizes under stringent conditions to at least about 12, 20, 25, 40, 50 or more consecutive nucleotides.

The polynucleotide sequence is useful as hybridization probes for determining the presence, level, form and distribution of nucleic acid expression. Accordingly, the probes can be used to detect the presence of, or to determine levels of, a specific nucleic acid molecule in cells, tissues, and in organisms. The nucleic acid whose level is determined can be DNA or RNA. Accordingly, probes corresponding to the peptides described herein can be used to assess expression and/or gene copy number in a given cell, tissue, or organism. These uses are relevant for detecting conditions involving an increase or decrease in enzyme protein expression contributing to sulfation activity towards the 16-androstene steroids.

The invention contemplates in vitro techniques for detection of mRNA such as Southern hybridizations, Northern hybridizations and in situ hybridizations.

The invention also provides vectors containing the nucleic acid molecule described herein. When the vector is a nucleic acid molecule, the nucleic acid molecule is covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the nucleic acid molecule. Alternatively, the vector may integrate into the host cell genome and produce additional copies of the nucleic acid molecules when the host cell replicates.

The invention provides vectors for the maintenance (cloning vectors) or vectors for expression (expression vectors) of the nucleic acid molecules. The vectors can function in prokaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, a trans-acting factor may be supplied by the host cell. Finally, a trans-acting factor can be produced from the vector itself. It is understood, however, that in some embodiments, transcription and/or translation of the nucleic acid molecules can occur in a cell-free system.

The regulatory sequence to which the nucleic acid molecule described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a nucleic acid molecule. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g., cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e., tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

As described herein, it may be desirable to express the peptide as a fusion protein. Accordingly, the invention provides fusion vectors that allow for the production of the peptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. Proteolytic enzymes include, but are not limited to, factor Xa, thrombin, and enteroenzyme. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

The vector containing the appropriate nucleic acid molecule can be introduced into an appropriate host cell for propagation or expression using well-known techniques. The invention therefore also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

Recombinant protein expression can be maximized in host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990)119-128). Alternatively, the sequence of the nucleic acid molecule of interest can be altered to provide preferential codon usage for a specific host cell, for example E. coli. (Wada et al., Nucleic Acids Res. 20:2111-2118 (1992)).

The nucleic acid molecule can also be expressed by expression vectors that are operative in yeast. Examples of vectors for expression in yeast e.g., S. cerevisiae include pYepSec1 (Baldari, et al., EMBO J. 6:229-234 (1987)), pMFa (Kujan et al., Cell 30:933-943(1982)), pJRY88 (Schultz et al., Gene 54:113-123 (1987)), and pYES2 (Invitrogen Corporation, San Diego, Calif.).

In certain embodiments of the invention, the nucleic acid molecule described herein is expressed in mammalian cells using mammalian expression vectors. Examples of mammalian expression vectors include pCDM8 (Seed, B. Nature 329:840(1987)) and pMT2PC (Kaufman et al., EMBO J. 6:187-195 (1987)).

The expression vectors listed herein are provided by way of example only of the well-known vectors available to those of ordinary skill in the art that would be useful to express the nucleic acid molecule. The person of ordinary skill in the art would be aware of other vectors suitable for maintenance propagation or expression of the nucleic acid molecule described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequence described herein are cloned into the vector in reverse orientation, but operably linked to a regulatory sequence that permits transcription of antisense RNA. Thus, an antisense transcript can be produced to all, or to a portion, of the nucleic acid molecule sequence described herein, including both coding and non-coding regions. Expression of this antisense RNA is subject to each of the parameters described above in relation to expression of the sense RNA (regulatory sequences, constitutive or inducible expression, tissue-specific expression).

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the nucleic acid molecule can be introduced either alone or with other nucleic acid molecules that are not related to the nucleic acid molecule such as those providing trans-acting factors for expression vectors. When more than one vector is introduced into a cell, the vectors can be introduced independently, co-introduced or joined to the nucleic acid molecule vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the nucleic acid molecules described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein. Where secretion of the peptide is desired, which is difficult to achieve with multi-transmembrane domain containing proteins such as enzymes, appropriate secretion signals are incorporated into the vector. The signal sequence can be endogenous to the peptides or heterologous to these peptides.

Where the peptide is not secreted into the medium, which is typically the case with enzymes, the protein can be isolated from the host-cell by standard disruption procedures, including freeze thaw, sonication, mechanical disruption, use of lysing agents and the like. The peptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the peptide described herein, the peptide can have various glycosylation patterns, depending upon the cell, or maybe non-glycosylated as when produced in bacteria. In addition, the peptide may include an initial modified methionine in some cases as a result of a host-mediated process.

The recombinant host cells expressing the peptide described herein have a variety of uses. The cells are useful for producing an enzyme protein or peptide that can be further purified to produce desired amounts of enzyme protein or fragments.

Host cells are also useful for conducting cell-based assays involving the enzyme protein or enzyme protein fragments, such as those described above as well as other formats known in the art. Thus, a recombinant host cell expressing a native enzyme protein is useful for assaying compounds that stimulate or enhance enzyme protein function.

Host cells are also useful for identifying enzyme protein mutants in which these functions are affected. If the mutants naturally occur and give rise to a condition such as boar taint, host cells containing the mutations are useful to assay compounds that have a desired effect on the mutant enzyme protein (for example, stimulating or inhibiting function) which may not be indicated by their effect on the native enzyme protein.

Genetically engineered host cells can be further used to produce non-human transgenic animals. A transgenic animal is preferably a mammal, for example a rodent, such as a rat or mouse or pig, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of an enzyme protein and identifying and evaluating modulators of enzyme protein activity. Other examples of transgenic animals include non-human primates, sheep, dogs, cows, goats, chickens, and amphibians. In a preferred embodiment, the transgenic animal is a pig.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the enzyme protein nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the enzyme protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. Such methods without undue experimentation are applicable to pig. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al. PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring born of this female foster animal will be a clone of the animal from which the cell, e.g., the somatic cell, is isolated.

Transgenic animals containing recombinant cells that express the peptides described herein are useful to conduct the assays described herein in an in vivo context. Accordingly, the various physiological factors that are present in vivo and that could effect substrate binding, enzyme protein activation, and signal transduction, may not be evident from in vitro cell-free or cell-based assays. Accordingly, it is useful to provide non-human transgenic animals to assay in vivo enzyme protein function, including substrate interaction, the effect of specific mutant enzyme proteins on enzyme protein function and substrate interaction, and the effect of chimeric enzyme proteins. It is also possible to assess the effect of null mutations, that is, mutations that substantially or completely eliminate one or more enzyme protein functions.

In another embodiment, the present invention provides a polynucleotide sequence that encodes a polypeptide that has been identified as being a member of the enzyme family of sulfotransferase proteins (the amino acid sequence of the porcine SULT2A1 cDNA is provided in FIG. 2 (SEQ ID NO:2).

The present invention further provides a protein that consists essentially of a protein encoded by the transcript/cDNA nucleic acid sequences shown in FIG. 2 (SEQ ID NO:2). A protein consists essentially of an amino acid sequence when such an amino acid sequence is present with only a few additional amino acid residues, for example from about 1 to about 100 or so additional residues, typically from 1 to about 20 additional residues in the final protein.

The present invention further provides a protein that comprises the amino acid sequence encoded by the transcript/cDNA nucleic acid sequences shown in FIG. 2 (SEQ ID NO:2). A protein comprises an amino acid sequence when the amino acid sequence is at least part of the final amino acid sequence of the protein. In such a fashion, the protein can be only the peptide or have additional amino acid molecules, such as amino acid residues. Such a protein can have a few additional amino acid residues or can comprise several hundred or more additional amino acids. The preferred classes of proteins that are comprised of the enzyme peptides of the present invention are the naturally occurring mature proteins.

The enzyme protein of the present invention can be attached to heterologous sequences to form chimeric or fusion proteins. Such chimeric and fusion proteins comprise an enzyme peptide operatively linked to a heterologous protein having an amino acid sequence not substantially homologous to the enzyme peptide. “Operatively linked” indicates that the enzyme peptide and the heterologous protein are fused in-frame. The heterologous protein can be fused to the N-terminus or C-terminus of the enzyme peptide.

In some uses, the fusion protein does not affect the activity of the enzyme peptide per se. For example, the fusion protein can include, but is not limited to, enzymatic fusion proteins, for example beta-galactosidase fusions, yeast two-hybrid GAL fusions, poly-His fusions, MYC-tagged, HI-tagged and Ig fusions. Such fusion proteins, particularly poly-His fusions, can facilitate the purification of recombinant enzyme peptide. In certain host cells (e.g., mammalian host cells), expression and/or secretion of a protein can be increased by using a heterologous signal sequence.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). An enzyme peptide-encoding nucleic acid can be cloned into such an expression vector such that the fusion moiety is linked in-frame to the enzyme peptide.

The present invention also provides and enables obvious variants of the amino acid sequence of the proteins of the present invention, such as naturally occurring mature forms of the peptide, allelic/sequence variants of the peptides, non-naturally occurring recombinantly derived variants of the peptides, and orthologs and paralogs of the peptides. Such variants can readily be generated using art-known techniques in the fields of recombinant nucleic acid technology and protein biochemistry. It is understood, however, that variants exclude any amino acid sequences disclosed prior to the invention.

Such variants can readily be identified/made using molecular techniques and the sequence information disclosed herein. Further, such variants can readily be distinguished from other peptides based on sequence and/or structural homology to the enzyme peptides of the present invention. The degree of homology/identity present will be based primarily on whether the peptide is a functional variant or non-functional variant, the amount of divergence present in the paralog family and the evolutionary distance between the orthologs.

The determination of the percent identity of two amino acid sequences or two nucleic acid sequences, is discussed supra the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. The comparison of sequences and determination of percent identity and similarity between two sequences is discussed in further detail supra.

It is contemplated that it is possible to modify the structure of a peptide having a function (e.g., SULT2A1 function) for such purposes as altering the biological activity. Such modified peptides are considered functional equivalents of peptides having an activity of SULT2A1 as defined herein. A modified peptide can be produced in which the nucleotide sequence encoding the polypeptide has been altered, such as by substitution, deletion, or addition. In particularly preferred embodiments, these modifications do not significantly reduce the biological activity of the modified SULT2A1. In other words, construct “X” can be evaluated in order to determine whether it is a member of the genus of modified or variant SULT2A1 of the present invention as defined functionally, rather than structurally. In preferred embodiments, the activity of variant SULT2A1 polypeptide is evaluated by methods described herein (e.g., the generation of transgenic animals).

Moreover, as described above, variant or polymorphic forms of SULT2A1 are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of SULT2A1 disclosed herein containing conservative replacements.

As used herein, a fragment comprises at least 8, 10, 12, 14, 16, or more contiguous amino acid residues from an enzyme peptide. Such fragments can be chosen based on the ability to retain one or more of the biological activities of the enzyme peptide or could be chosen for the ability to perform a function, e.g. bind a substrate or act as an immunogen. Particularly important fragments are biologically active fragments, peptides that are, for example, about 8 or more amino acids in length. Such fragments will typically comprise a domain or motif of the enzyme peptide, e.g., active site, a transmembrane domain or a substrate-binding domain. Further, possible fragments include, but are not limited to, domain or motif containing fragments, soluble peptide fragments, and fragments containing immunogenic structures. Predicted domains and functional sites are readily identifiable by computer programs well known and readily available to those of skill in the art (e.g., PROSITE analysis).

Polypeptides often contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in enzyme peptides are described in basic texts, detailed monographs, and the research literature, and they are well known to those of skill in the art).

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

Accordingly, the enzyme protein of the present invention also encompasses derivatives or analogs in which a substituted amino acid residue is not one encoded by the genetic code, in which a substituent group is included, in which the mature enzyme peptide is fused with another compound, such as a compound to increase the half-life of the enzyme peptide (for example, polyethylene glycol), or in which the additional amino acids are fused to the mature enzyme peptide, such as a leader or secretory sequence or a sequence for purification of the mature enzyme peptide or a pro-protein sequence.

The proteins of the present invention can be used in substantial and specific assays related to the function; to raise antibodies or to elicit another immune response; as a reagent (including the labeled reagent) in assays designed to quantitatively determine levels of the protein (or its binding partner or ligand) in biological samples; and as markers for tissues in which the corresponding protein is preferentially expressed (either constitutively or at a particular stage of tissue differentiation or development or in a disease state). Where the protein binds or potentially binds to another protein or ligand (such as, for example, in an enzyme-effector protein interaction or enzyme-ligand interaction), the protein can be used to identify the binding partner/ligand so as to develop a system to identify inhibitors of the binding interaction. Any or all of these uses are capable of being developed into reagent grade or kit format for commercialization as commercial products.

Methods for performing the uses listed above are well known to those skilled in the art. References disclosing such methods include “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The potential uses of the proteins of the present invention are based primarily on the source of the protein as well as the class/action of the protein. For example, SULT2A1 enzyme isolated from pigs serve as targets for identifying agents for reducing boar taint in pigs.

The protein of the present invention (including variants and fragments) is also useful for biological assays related to enzymes that are related to members of the sulfotransferase family. Such assays involve any of the known enzyme functions or activities or properties useful for alleviating enzyme-related conditions that are specific for the sulfotransferase enzymes, particularly in cells and tissues that express the enzyme.

The present invention also contemplates drug screening assays, in cell-based or cell-free systems. Cell-based systems can be native, i.e., cells that normally express the enzyme, as a biopsy or expanded in cell culture.

To perform cell free drug screening assays, it is sometimes desirable to immobilize either the enzyme protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of enzyme-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of an enzyme-binding protein and a candidate compound are incubated in the enzyme protein-presenting wells and the amount of complex trapped in the well can be quantitated. Methods for detecting such complexes, in addition to those described above for the GST-immobilized complexes, include immunodetection of complexes using antibodies reactive with the enzyme protein target molecule, or which are reactive with enzyme protein and compete with the target molecule, as well as enzyme-linked assays which rely on detecting an enzymatic activity associated with the target molecule.

The polypeptides can be used to identify compounds that modulate enzyme activity of the protein in its natural state or an altered form that catalyzes the sulfoconjugation of 5α-androstenone whose concentration in adipose tissue is influenced by the amount of unconjugated steroid that is present in the circulation. It is contemplated by the present invention that the enzymes and appropriate variants and fragments can be used in high-throughput screens to assay candidate compounds for the ability to bind to the enzyme. These compounds can be further screened against a functional enzyme to determine the effect of the compound on the enzyme activity. Further, these compounds can be tested in animal or invertebrate systems to determine activity/effectiveness. Compounds can be identified that activate (agonist) or inactivate (antagonist) the enzyme to a desired degree.

Further, the protein of the present invention can be used to screen a compound for the ability to stimulate or inhibit interaction between the enzyme protein and a molecule that normally interacts with the enzyme protein, e.g. a substrate or a component of the signal pathway that the enzyme protein normally interacts (for example, another enzyme). Such assays typically include the steps of combining the enzyme protein with a candidate compound under conditions that allow the enzyme protein, or fragment, to interact with the target molecule, and to detect the formation of a complex between the protein and the target or to detect the biochemical consequence of the interaction with the enzyme protein and the target, such as any of the associated effects of signal transduction such as protein phosphorylation, cAMP turnover, and adenylate cyclase activation, etc.

Candidate compounds include, for example, 1) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L-configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The peptides of the present invention may also provide targets for regulating protein activity in an animal, e.g., a pig. When a mutation is functionally correlated to the activity or level of the protein, the protein also provides a target for diagnosing active protein activity, or predisposition to boar taint, in an animal, e.g., a pig, having a variant protein. Thus, the peptide can be isolated from a biological sample and assayed for the presence of a genetic mutation that results in aberrant peptide. This includes amino acid substitution, deletion, insertion, rearrangement, (as the result of aberrant splicing events), and inappropriate post-translational modification. Analytic methods include altered electrophoretic mobility, altered tryptic peptide digest, altered enzyme activity in cell-based or cell-free assay, alteration in substrate or antibody-binding pattern, altered isoelectric point, direct amino acid sequencing, and any other of the known assay techniques useful for detecting mutations in a protein. Such an assay can be provided in a single detection format or a multi-detection format such as an antibody chip array.

In vitro techniques for detection of peptide include enzyme linked immunosorbent assays (ELISAs), Western blots, immunoprecipitations and immunofluorescence using a detection reagent, such as an antibody or protein binding agent. Alternatively, the peptide can be detected in vivo in a subject by introducing into the subject a labeled anti-peptide antibody or other types of detection agent. For example, the antibody can be labeled with a radioactive marker whose presence and location in a subject can be detected by standard imaging techniques. Particularly useful are methods that detect the allelic variant of a peptide expressed in a subject and methods which detect fragments of a peptide in a sample. All methods are well known to those of skill in the art.

In other embodiment, the invention provides antibodies that selectively bind to the polypeptide of the present invention, as well as variants and fragments thereof. An antibody selectively binds a target peptide when it binds the target peptide and does not significantly bind to unrelated proteins. An antibody is still considered to selectively bind a peptide even if it also binds to other proteins that are not substantially homologous with the target peptide so long as such proteins share homology with a fragment or domain of the peptide target of the antibody. In this case, it would be understood that antibody binding to the peptide is still selective despite some degree of cross-reactivity.

Antibodies of the invention (e.g., a monoclonal antibody) can be used to isolate wild-type or a variant polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a wild-type or a variant polypeptide of the invention can be used to detect the polypeptide (e.g., in a cellular lysate, cell supernatant, or tissue sample) in order to evaluate the abundance and pattern of activity or expression of the polypeptide. Antibodies can also be used to monitor protein levels in tissue.

Antibodies to the polypeptide or variant of the invention can be prepared by well known methods using a purified protein according to the invention or a (synthetic) fragment derived therefrom as an antigen. In a preferred embodiment of the invention, the antibody is a monoclonal antibody or a polyclonal antibody that specifically binds the polypeptide. Such antibodies also include bispecific antibody, synthetic antibody, antibody fragment, such as Fab, Fv or scFv fragments etc., or a chemically modified derivative of any of these.

Monoclonal antibodies can be prepared, for example, by the techniques as originally described in Kohler and Milstein, Nature 256:495-497 (1975), and Galfre, Meth. Enzymol. 73 (1981), 3, which comprise the fusion of mouse myeloma cells to spleen cells derived from immunized mammals. Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, e.g., Kohler and Milstein (1975). The technology for producing hybridomas is well known in the art (see e,g., Current Protocols in Immunology (1994) Coligan et al. (eds.) John Wiley & Sons, Inc., New York, N.Y., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al., Monoclonal Antibodies in Cancer Therapy (1985) Allen R. Bliss, Inc., pages 77-96), and screening of combinatorial antibody libraries (Huse et al., Science 246, 1275 (1989)). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Alternative to preparing monoclonal antibody-secreting hybridomas, a monoclonal antibody to a wild-type or a variant polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurJZAP.™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Recombinant antibodies, such as chimeric and humanized antibodies, which can be made using standard recombinant DNA techniques, are within the scope of the invention. Conventional methods may be used to make chimeric antibodies containing the immunoglobulin variable region which recognizes a protein of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Detection of antibodies of the invention can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β (beta)-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the variant polypeptides of the invention as well as for the monitoring of the presence of said variant polypeptides, for example, in recombinant organisms, and for the identification of compounds interacting with the proteins according to the invention.

The antibodies can be used to isolate the protein of the present invention by standard techniques, such as affinity chromatography or immunoprecipitation. The antibodies can facilitate the purification of the natural protein from cells and recombinantly produced protein expressed in host cells. In addition, such antibodies are useful to detect the presence of one of the proteins of the present invention in cells or tissues to determine the pattern of expression of the protein among various tissues in an organism and over the course of normal development. Further, such antibodies can be used to detect protein in situ, in vitro, or in a cell lysate or supernatant in order to evaluate the abundance and pattern of expression. Also, such antibodies can be used to assess 5α-androstenone tissue distribution during development or progression of a biological condition. Antibody detection of circulating fragments of the full length protein can be used to identify turnover.

The invention also encompasses kits for using antibodies to detect the presence of a protein in a biological sample. The kit can comprise antibodies such as a labeled or labelable antibody and a compound or agent for detecting protein in a biological sample; means for determining the amount of protein in the sample; means for comparing the amount of protein in the sample with a standard; and instructions for use. Such a kit can be supplied to detect a single protein or epitope or can be configured to detect one of a multitude of epitopes, such as in an antibody detection array. Antibody arrays are contemplated by the present invention.

Nucleic acid expression assays are useful for drug screening to identify compounds that modulate enzyme nucleic acid expression. The invention also relates to methods for reducing boar taint by manipulating the SULT2A1 polynucleotide sequence and/or its gene product.

Accordingly in another embodiment, the invention provides a method for identifying a compound that can be used to regulate 16-androstene steroids metabolism in a pig. Substances that regulate 16-androstene steroid metabolism include substances that modulate Phase I hydrolysis, reduction and oxidation or Phase II conjugation reactions involved in 16-androstene steroids metabolism. Preferably, the substances enhance the activity or expression of a hydroxysteroid sulfotransferase, SULT2A1 because as described herein, low SULT2A1 activity results in decreased levels of the sulfoconjugated form 5α-androstenone so that more of the unconjugated form can accumulate in adipose tissue in high boar taint pigs.

In one embodiment of the present invention, a method is provided for screening for a substance that enhances 16-androstene steroid metabolism in a pig by enhancing SULT2A1 activity comprising reacting a substrate of SULT2A1 and SULT2A1, in the presence of a test substance, under conditions such that SULT2A1 is capable of converting the substrate into a reaction product; assaying for reaction product, unreacted substrate or unreacted SULT2A1; and comparing to controls to determine if the test substance selectively enhances SULT2A1 activity and thereby is capable of enhancing 16-androstene steroid metabolism in a pig. Suitable controls include female pigs and male pigs that are known to have boar taint.

Substrates of SULT2A1 which may be used in the method of the invention for example include dehydroepiandrosterone (DHEA) and steroids that contain 3β (beta), 3α (alpha), and 17β hydroxyl groups. The induction of SULT2A1 sulfotransferase activity can be measured using a variety of techniques known in the art. For example levels of a hydroxysteroid sulfotransferase can be measured using Western blotting. Other methods include measuring the biological activity of the enzyme.

Thus, modulators of enzyme gene expression can be identified in a method wherein a cell is contacted with a candidate compound and the expression of mRNA determined. The level of expression of enzyme mRNA in the presence of the candidate compound is compared to the level of expression of enzyme mRNA in the absence of the candidate compound. The candidate compound can then be identified as a modulator of nucleic acid expression based on this comparison and be used, for example to reduce boar taint characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Alternatively, a modulator for enzyme nucleic acid expression can be a small molecule or drug identified using the screening assays described herein as long as the drug or small molecule enhances the enzyme nucleic acid expression in the cells and tissues that express the protein.

The polynucleotide disclosed herein is also useful for monitoring the effectiveness of modulating compounds on the expression or activity of the enzyme gene in metabolism studies for monitoring the accumulation of 5α-androstenone in fat. Thus, the gene expression pattern can serve as a barometer for the continuing effectiveness of treatment with the compound. The gene expression pattern can also serve as a marker indicative of a physiological response of the affected cells to the compound. Accordingly, such monitoring would allow either increased administration of the compound or the administration of alternative compounds to which the animal has not become resistant. Similarly, if the level of nucleic acid expression falls below a desirable level, administration of the compound could be commensurately decreased.

In another embodiment, the invention provides a method for reducing or preventing boar taint comprising enhancing the metabolism of 5α-androstenone in a pig comprising administering to a pig a substance, in sufficient quantity or a therapeutically effective amount and under appropriate conditions, that induces or increases or enhances the expression of a SULT2A1 polynucleotide sequence to induce or increase or enhance or up regulate the expression of the amino acid sequence.

In another embodiment, the invention provides a method for screening for a substance that enhances 5α-androstene metabolism by enhancing transcription and/or translation of the nucleotide sequence encoding SULT2A1 comprising culturing a host cell comprising a nucleic acid molecule containing a nucleic acid sequence encoding SULT2A1 and the necessary elements for the transcription or translation of the nucleic acid sequence, and optionally a reporter gene, in the presence of a test substance; and comparing the level of expression of SULT2A1, or the expression of the protein encoded by the reporter gene with a control cell transfected with a nucleic acid molecule in the absence of the test substance.

A host cell for use in the method of the invention may be prepared by transfecting a suitable host with a nucleic acid molecule comprising a nucleic acid sequence encoding the appropriate enzyme. Suitable transcription and translation elements may be derived from a variety of sources, including bacterial, fungal, viral, mammalian, or insect genes. Selection of appropriate transcription and translation elements is dependent on the host cell chosen, and may be readily accomplished by one of ordinary skill in the art. Examples of such elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, and sequences conferring inducibility of transcription may be incorporated into the expression vector. It will also be appreciated that the necessary transcription and translation elements may be supplied by the native gene of the enzyme and/or its flanking sequences.

Examples of reporter genes are genes encoding a protein such as β (beta)-galactosidase, chloramphenicol acetyltransferase, firefly luciferase, or an immunoglobulin or portion thereof such as the Fc portion of an immunoglobulin, preferably IgG. Transcription of the reporter gene is monitored by changes in the concentration of the reporter protein such as β (beta)-galactosidase, chloramphenicol aceryltransferase, or firefly luciferase. This makes it possible to visualize and assay for expression of the enzyme and in particular to determine the effect of a substance on expression of enzyme.

Suitable host cells are disclosed earlier herein. Host cells which are commercially available may also be used in the method of the invention. For example, the h2A3 and h2B6 cell lines available from Gentest Corporation are suitable for the screening methods of the invention.

In yet another embodiment, the invention also contemplates genetic markers that may be found in the porcine SULT2A1 gene. One mutation disclosed herein, a cytosine to a thymine found within the coding region at bp position 219 did not affect the amino acid sequence of the enzyme. It is expected that other polymorphisms may be found without undue experimentation within the SULT2A1 gene. Such genetic markers would be useful, for example, in identifying animals that have a decreased genetic capacity to accumulate 5α-androstenone in fat while maintaining the normal levels of testicular steroids that are characteristic of intact male pigs. While this particular polymorphism identified did not cause a change in amino acid sequence and is therefore a silent mutation such silent mutations are often linked to other polymorphic seqeucnes that can affect SULT2A1 activity,indirectly or may have effects on other realted genes. In yet another example, a silent polymorphic mutation may be in the promoter region of the gene and thus affect SULT2A1 activity. It is expected that other polymorphisms found in the SULT2A1 gene will have nucleotide changes that result in amino acid substitutions, additions, deletions, fusions and truncations in the polypeptide encoded by the polynucleotide sequence wherein the polypeptide can be either fully functionally or can lack function in one or more sulfotransferase activities, e.g., ability to bind substrate, ability to catalyze the sulfonation of hydroxyl groups in steroids, ability to mediate signaling. Fully functional variants typically contain only conservative variation or variation in non-critical residues or in non-critical regions. Functional variants can also contain substitution of similar amino acids that result in no change or an insignificant change in function. Non-functional variants typically contain one or more non-conservative amino acid substitutions, deletions, insertions, inversions, or truncation or a substitution in a critical residue or critical region. Amino acids that are essential for function can be identified by methods known in the art, such as site-directed mutagenesis.

Substances which enhance 5α-androstenone metabolism described in detail herein or substances identified using the methods of the invention which selectively enhance SULT2A1 activity may be incorporated into pharmaceutical compositions. Therefore, the invention provides a pharmaceutical composition for use in reducing boar taint comprising an effective amount of one or more substances which enhance 5α-androstenone metabolism and/or a pharmaceutically acceptable carrier, diluent, or excipient. In one embodiment, the present invention provides a pharmaceutical composition comprising an effective amount of the substance which is selected from the group consisting of: a substance that increases the activity of the SULT2A1 enzyme; and (b) a substance that induces or increases the expression of the SULT2A1 gene.

The substances for the present invention can be administered for oral, topical, rectal, parenteral, local, inhalant or intracerebral use. Preferably, the active substances are administered orally (in the food or drink) or as an injectable formulation.

In the methods of the present invention, the substances described in detail herein and identified using the method of the invention form the active ingredient, and are typically administered in admixture with suitable pharmaceutical diluents, excipients, or carriers suitably selected witch respect to the intended form of administration, that is, oral tablets, capsules, elixirs, syrups and the like, consistent with conventional veterinary practices.

For example, for oral administration the active ingredients may be prepared in the form of a tablet or capsule for inclusion in the food or drink. In such a case, the active substances can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral active substances can be combined with any oral, non-toxic, pharmaceutically acceptable inert carrier such as ethanol, glycerol, water, and the like. Suitable binders, lubricants, disintegrating agents, and coloring agents can also be incorporated into the dosage form if desired or necessary. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Suitable lubricants used in these dosage forms include sodium oleate, sodium stearate, magnesium stearate, sodium benzoate, sodium acetate, sodium chloride, and the like. Examples of disintegrators include starch, methyl cellulose, agar, bentonite, xanthan gum, and the like.

Gelatin capsules may contain the active substance and powdered carriers, such as lactose, starch, cellulose derivatives, magnesium stearate, stearic acid, and the like. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract. Liquid dosage forms for oral administration may contain coloring and flavoring agents to increase acceptance.

Water, a suitable oil, saline, aqueous dextrose, and related sugar solutions and glycols such as propylene glycol or polyethylene glycols, may be used as carriers for parenteral solutions. Such solutions also preferably contain a water soluble salt of the active ingredient, suitable stabilizing agents, and if necessary, buffer substances. Suitable stabilizing agents include antioxidizing agents such as sodium bisulfate, sodium sulfite, or ascorbic acid, either alone or combined, citric acid and its salts and sodium EDTA. Parenteral solutions may also contain preservatives, such as benzalkonium chloride, methyl- or propyl-paraben, and chlorobutanol.

The substances described in detail herein and identified using the methods of the invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Substances described in detail herein and identified using the methods of the invention may also be coupled with soluble polymers which are targetable drug carriers. Examples of such polymers include polyvinylpyrrolidone, pyran copolymer, polyhydroxypropyl-methacrylamideph-enol, polyhydroxyethyl-aspartamidephenol, or polyethyleneoxide-polylysine substituted with palmitoyl residues. The substances may also be coupled to biodegradable polymers useful in achieving controlled release of a drug. Suitable polymers include polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Suitable pharmaceutical carriers and methods of preparing pharmaceutical dosage forms are described in Remington's Pharmaceutical Sciences Mack Publishing Company, a standard reference text in this field.

More than one substance described in detail herein or identified using the methods of the invention may be used to enhance metabolism of 16-androstene steroids. In such cases the substances can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chosen route of administration and standard pharmaceutical practice as described herein.

The agents are administered in a therapeutically effective amount. The amount of agents which will be therapeutically effective in the treatment of a particular condition, boar taint, will depend on the nature of the condition, and can be determined by standard veterinary techniques. In addition, in vitro or in vivo assays may optionally be employed to help identify optimal dosage ranges. The precise dose to be employed in the formulation will also depend on the route of administration, and should be decided according to the judgment of a practitioner and each animal's circumstances. As is well known in the veterinary arts, dosages for any one animal depends upon many factors, including the animal's size, body surface area, age, the particular compound to be administered, time and route of administration, general health, and other drugs being administered concurrently. Progress can be monitored by periodic assessment. Effective doses may be extrapolated from dose-response curves derived from in vitro model test systems.

The identification of the existence of a polymorphism within a gene is often made by a single base alternative that results in a restriction site in certain allelic forms. A certain allele; however, as discussed herein, may have a number of base changes associated with it that could be assayed for which are indicative of the same polymorphism (allele). Further, other genetic markers or genes may be linked to the polymorphisms disclosed herein so that assays may involve identification of other genes or gene fragments, but which ultimately rely upon genetic characterization of animals for the same polymorphism. Any assays which sorts and identifies animals based upon the allelic differences disclosed herein are intended to be included within the scope of this invention.

Animals carrying mutation(s) in the enzyme gene can be detected at the nucleic acid level by a variety of techniques. The gene encoding the novel enzyme of the present invention is located on a genome component that has been mapped to porcine chromosome. Genomic DNA can be analyzed directly or can be amplified by using PCR prior to analysis. RNA or cDNA can be used in the same way.

Any method of identifying the presence or absence of these markers may be used, including, for example, single-strand conformation polymorphism (SSCP) analysis Fischer et al. (1983) Proc. Natl. Acad. Sci. USA 80:1579-1583, Orita et al. (1989) Genomics 5:874-879, base excision sequence scanning (BESS), RFLP analysis, heteroduplex analysis, denaturing gradient gel electrophoresis, and temperature gradient electrophoresis, allelic PCR, ligase chain reaction direct sequencing, mini sequencing, nucleic acid hybridization, micro-array-type detection of genes encoding enzymes involved in 16-androstene steroid metabolism. The polymorphism may or may not be the causative mutation but will be indicative of the presence of this change and one may assay for the genetic or protein bases for the phenotypic difference. In a preferred method, single-strand conformation polymorphism (SSCP) analysis is used to identifying the presence or absence of these markers.

The following assays are useful in the present invention. In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W. H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the gene as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

Other methods of nucleic acid analysis can be used to detect polymorphisms in SULT2A1. Representative methods include direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al. U.S. Pat. No. 5,288,644); automated fluorescent sequencing; clamped denaturing gel electrophoresis (CDGE); mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); chemical mismatch cleavage (CMC) (Cotton et al. (1985) Proc. Natl. Acad. Sci. USA 85:4397-4401); RNase protection assays (Myers, R. M. et al. (1985) Science 230:1242); amplified fragment-length polymorphism (AFLP) Vos et al. (1995) Nucleic Acids Res 23:4407-4414; microsatellite or single-sequence repeat (SSR) Weber J L and May P E (1989) Am J Hum Genet 44:388-396; rapid-amplified polymorphic DNA (RAPD) Williams et al. (1990) Nucleic Acids Res 18:6531-6535; sequence tagged site (STS) Olson et al. (1989) Science 245:1434-1435; genetic-bit analysis (GBA) Nikiforov et al (1994) Nucleic Acids Res 22:4167-4175; nick-translation PCR (e.g., TAQMAN™) Lee et al. (1993) Nucleic Acids Res 21:3761-3766; and allele-specific hybridization (ASH) Wallace et al. (1979) Nucleic Acids Res 6:3543-3557, (Sheldon et al. (1993) Clinical Chemistry 39(4):718-719); use of polypeptides which recognize nucleotide mismatches, such as E. coli mutS protein, for example. Each technology has its own particular basis for detecting polymorphisms in DNA sequence.

The following is a general overview of some techniques which can be used to assay for polymorphisms in the polynucleotide sequence of the invention.

PCR Amplification

The most common means for amplification is polymerase chain reaction (PCR), as described in U.S. Pat. Nos. 4,683,195; 4,683,202; and 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on a sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, and 0.5% NP40 supplemented with 100 μg/ml of proteinase K. After incubating at 56° C. for 2 hr the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed so that under low stringency, they will bind to both polymorphic forms of the allele, but at high stringency, bind to the allele to which they correspond. Alternatively, stringency conditions can be devised in which an essentially binary response is obtained, i.e., an ASO corresponding to a variant form of the target gene will hybridize to that allele, and not to the wild-type allele.

Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, “Principles and Applications for DNA Amplification”, W. H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tₘ's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or alleles at the chosen boar taint loci can be differentiated using single-strand conformation polymorphism analysis, which identifies base differences by alteration in electrophoretic migration of single-stranded PCR products, as described in Orita et al., Proc. Nat. Acad. Sci. 85:2766-2770 (1989). Amplified PCR products can be generated as described above, and heated or otherwise denatured, to form single-stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with polymorphisms.

Non-Gel Systems

Other possible techniques include non-gel systems such as TAQMAN™ (Perkin Elmer). In this system, oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e., there is a mismatch of some form, the cleavage of the dye does not take place. Thus, only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay, which includes isothermic amplification that relies on a catalytic release of fluorescence. See Third Wave Technology at www.twt.com.

Non-PCR Based DNA Diagnostics

The identification of a DNA sequence linked to sequences encoding enzymes involved in 16-androstene steroid metabolism can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with P³² or S³⁵. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the porcine chromosome where the sulfotransferase gene or other gene involved in 16-androstene steroid metabolism resides, and thus defining a genetic marker linked to the gene, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

According to the invention, polymorphisms in genes encoding enzymes involved in 16-androstene steroid metabolism have been identified which have an association with boar taint. The presence or absence of the markers, in one embodiment may be assayed by PCR-RFLP analysis using the restriction endonucleases and amplification primers may be designed using analogous human, pig or other sequences due to the high homology in the region surrounding the polymorphisms, or may be designed using known gene sequence data as exemplified in GenBank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), Short Protocols in Molecular Biology, 4th Edition, John Wiley and Sons (1999).

The following is a brief description of primer design. Generally the primers used for the assays of the invention will flank nt 546 on each side, one forward and one reverse.

Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above. If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

The methods and materials of the invention may also be used more generally to evaluate pig DNA, genetically type individual pigs, and detect genetic differences in pigs. In particular, a sample of pig genomic DNA may be evaluated by reference to one or more controls to determine if a polymorphism in the particular gene is present. Preferably, RFLP analysis is performed with respect to the pig gene, and the results are compared with a control. The control is the result of a RFLP analysis of the pig gene of a different pig where the polymorphism(s) of the pig gene is/are known. Similarly, the genotype of a pig may be determined by obtaining a sample of its genomic DNA, conducting RFLP analysis of the gene in the DNA, and comparing the results with a control. Again, the control is the result of RFLP analysis of the gene of a different pig. The results genetically type the pig by specifying the polymorphism(s) in its genes. Finally, genetic differences among pigs can be detected by obtaining samples of the genomic DNA from at least two pigs, identifying the presence or absence of a polymorphism in the gene, and comparing the results.

These assays are useful for identifying the genetic markers relating to boar taint, as discussed above, for identifying other polymorphisms in the genes encoding enzymes involved in 16-androstene steroid metabolism and for the general scientific analysis of pig genotypes and phenotypes.

The examples and methods herein disclose certain gene(s) which has been identified to have a polymorphism(s) which is associated either positively or negatively with a beneficial trait that will have an effect on boar taint for animals carrying this polymorphism. The identification of the existence of a polymorphism within a gene is often made by a single base alternative that results in a restriction site in certain allelic forms. A certain allele, however, as demonstrated and discussed herein, may have a number of base changes associated with it that could be assayed for which are indicative of the same polymorphism (allele). Further, other genetic markers or genes may be linked to the polymorphisms disclosed herein so that assays may involve identification of other genes or gene fragments, but which ultimately rely upon genetic characterization of animals for the same polymorphism. Any assays which sorts and identifies animals based upon the allelic differences disclosed herein are intended to be included within the scope of this invention.

One of skill in the art, once a polymorphism has been identified and a correlation to a particular trait established will understand that there are many ways to genotype animals for this polymorphism. The design of such alternative tests merely represents optimization of parameters known to those of skill in the art and is intended to be within the scope of this invention as fully described herein.

It should be understood that the detailed description and the specific examples while indicating preferred embodiments of the invention are given by way of illustration only, since various changes and modifications within the spirit and scope of the invention will become apparent to those skilled in the art from this detailed description.

### EXAMPLES

### Example 1

### Testicular Sulfoconjugation of the 16-Androstene Steroids by Hydroxysteroid Sulfotransferase: Its Impact on the Concentrations of 5α-Androstenone in Plasma and Fat of the Mature Domestic Boar

The 16-androstene steroids, specifically, 5α-androst-16-en-3α-ol (3α-androstenol), 5α-androst-16-en-3β-ol (3β-androstenol), and 5α-androst-16-en-3-one (5α-androstenone), have recently been shown to be sulfoconjugated by the Leydig cells of the mature boar (Philip A. Sinclair, 16-Androstene Steroid Metabolism and Its Impact on the Development of Boar Taint (2004) (PhD thesis, Univ. of Guelph). 5α-androstenone is mostly known for its involvement in boar taint (Gower, 1972; Patterson, 1968) which is the unpleasant odor that is associated with boar fat when it is heated.

A large proportion of steroids secreted from the boar testes are present in their sulfoconjugated form (Raeside et al., 1989; Raeside and Renaud, 1983; Tan and Raeside, 1980). In fact, the concentrations of many steroid sulfates in plasma are substantially higher than that of their unconjugated forms (Booth, 1983; Raeside and Howells, 1971; Tan and Raeside, 1980). This is due to the high levels of steroid sulfotransferase enzymes that are present in the Leydig cells (Hobkirk, 1985; Hobkirk et al., 1989; Raeside and Renaud, 1983). These enzymes include hydroxysteroid sulfotransferase (HST), estrogen sulfotransferase (EST), and phenol sulfotransferase (PST) (Baranczyk-Kuzma and Ciszewska-Pilczynska, 1989; Hobkirk, 1985). The major substrate for HST is dehydroepiandrosterone (DHEA); however, HST can also act on 3β, 3α, and some 17β-hydroxy steroids (Falany et al., 1989; Pedersen et al., 2000; Strott S. A., 1996). EST and PST act upon the hydroxyl groups of estrogens (Strott S. A., 1996). EST is also capable of sulfating hydroxysteroids such as DHEA and pregnenolone to a certain extent (Falany et al., 1994; Negishi et al., 2001).

The addition of a highly charged sulfate group greatly increases the polarity of a steroid (Jakoby et al., 1980; Strott S. A., 1996). This has important implications with respect to the accumulation of 5α-androstenone in adipose tissue.

Materials and Methods

Animals and Sampling

A total of 25 Yorkshire boars (group A), 175±6 days of age, were obtained from the Arkell Swine Research Station at the University of Guelph. In selecting these animals, physiological maturity was estimated by plasma testicular steroid concentrations, specifically estrone sulfate (E1S), and bulbourethral gland size (Allrich et al., 1982; Schwarzenberger et al., 1993; Sinclair et al., 2001 a). Mature animals were further screened based on total plasma 5α-androstenone levels. A cut-off level of 15 ng/mL was used, as it has been demonstrated that below this level animals do not have the capacity to develop boar taint (Sinclair et al., 2001 a). Blood samples were taken from the orbital sinus, centrifuged at 4° C. to collect plasma, and stored at −20° C. until extraction and analysis for steroid concentrations.

Testicular vein blood samples were taken from an additional 20 physiologically mature Yorkshire boars (Group B), >200 days of age, in order to determine the proportion of sulfoconjugated 5α-androstenone secreted from the testes. Blood was collected from veins on the surface of the testes as described by Raeside et al., 1989. The samples were centrifuged at 4° C. to collect serum and stored at −20° C. until extraction and analysis.

After the slaughter of group A boars, bulbourethral glands were removed and measured for length. A backfat sample was removed from the midline on the point of the 11^{th} rib and frozen at −20° C. until assayed for 5α-androstenone. Testes samples were obtained from five randomly selected animals immediately after slaughter. Testes were transported to the laboratory within five minutes of their removal from the boar. One testis was dissected free from the epididymis, cut longitudinally, and decapsulated. The tissue was sliced and 100 g of tissue was incubated for 12 min in a shaking water bath at 37° C. with 1 mg/mL collagenase (type 1A), 50 μg/mL trypsin inhibitor, and 50 μg/mL DNase in 250 mL of Williams Media E containing 1 g/L bovine serum albumin and 0.1 g/L L-glutamine. Purified Leydig cells were obtained by layering the collagenase dispersed cells onto discontinuous Percoll gradients (Raeside and Renaud, 1983). Cell viability was determined with a trypan blue exclusion test. The typical viability of the isolated Leydig cells was 90%.

Inhibition Studies

Purified Leydig cells were resuspended (50×10⁶ cells/incubation) into a final volume of 25 ml of Williams Media E mixture. Radiolabelled [7-³H(N)]-pregnenolone (3.4μCi/μmol) was added as a substrate to give a final concentration of 0.4 μmol/incubation. Leydig cells were incubated for a total of 8 hours at 37° C. under 95%:5% CO₂ atmosphere at in a Dubnoff shaking waterbath in the presence of 0, 5, 10, 50, or 100 μM of various sulfotransferase inhibitors. 8 hour incubations were used, as this is an optimal time-point for steroid conjugation under these conditions. Triethylamine, was used as an as an HST-inhibitor (Matsui et al., 1993). Pentachlorophenol (PCP) was used as a phenol-sulfotransferase-inhibitor (Boles and Klaassen, 1998; Fayz et al., 1984; Meerman et al., 1983); however, PCP does not inhibit HST (Okuda et al., 1989; Singer et al., 1984). Estrone was used as an EST-inhibitor. The sulfotransferase activity towards 5α-androstenone was determined by HPLC analysis of extracted 5 ml media aliquots.

Steroid Extraction and Purification

Prior to analysis, conjugated steroids were separated from unconjugated steroids with the use of Sep-Pak C₁₈ solid-phase chromatography cartridges as described previously (Raeside et al., 1997). After separation, the conjugate fraction was hydrolyzed overnight in trifluroacetic acid/ethyl acetate (1/100 v/v) at 45° C. in order to liberate the sulfoconjugated steroids (Raeside et al., 1999b). The solvolyzed steroids were then re-extracted by solid-phase chromatography. Enzyme hydrolysis was performed on the remaining conjugated material present after solvolysis with 1250 units of β-glucuronidase (type B-1, from bovine liver), and incubated overnight at 37° C. (Raeside et al., 1999a; Raeside et al., 1999b).

The identity of 16-androstene steroids extracted from the plasma was confirmed by HPLC purification followed by identification with GC-MS as outlined below. The unconjugated and hydrolyzed steroids were purified by HPLC using a modification of our previous methods (Bonneau et al., 1992). A Phenomenex 5μ C18 ODS HPLC column (250×4.6 mm) was used with an 85% acetonitrile: 15% H₂O mobile phase delivered isocratically at 0.7 ml/min. In this system, the 16-androstene steroids elute between 12 and 18 min, with 5α-androstenone eluting at 17.7 minutes. This purified fraction was evaporated to dryness under nitrogen at 45° C. and prepared for GC-MS.

Biochemical Analyses

Fat and extracted plasma samples were analyzed for 5α-androstenone with an ELISA method, modified after that described by Claus et al (Claus et al., 1988) and described previously (Squires and Lundstrom, 1997). Radioimmunoassay was used for measurements of E1S in plasma samples (Schwarzenberger et al., 1993).

Before GC-MS analysis, the purified 16-androstenes were derivatized as 0-methyloxime (MO) and trimethylsilyl (TMS) ethers (Khalil and Lawson, 1983), and subsequently purified using Lipidex 5000 column chromatography (Khalil et al., 1993). The eluates were evaporated to dryness under nitrogen at 45° C. and reconstituted in 200 ul of hexane/hexamethyldisilazane (20/1 v/v). Steroid derivatives were analyzed using a Hewlett Packard 6890 gas chromatography system equipped with a HP-1 capillary column linked to a Hewlett-Packard 5973N mass selective detector as described supra. To identify the steroids, the ion spectra were compared to those produced by the authentic steroid standards (Kwan et al., 1992).

The unconjugated and hydrolyzed steroids from the inhibition studies were analyzed by HPLC using the same protocol as stated above; however; radiolabelled steroids were measured on-line with a Canberra-Packard 500TR flow scintillation analyzer.

Statistical Analyses

Pearson correlation coefficients were calculated for the following measures: 1) plasma concentrations of sulfoconjugated 5α-androstenone vs. fat concentrations of 5α-androstenone, and 2) plasma concentrations of unconjugated 5α-androstenone vs. fat concentrations of 5α-androstenone (SAS Inst. Inc.).

Results

Proportions of Sulfoconjugated 5α-androstenone in Blood

Analysis of the unconjugated and sulfoconjugated fractions of peripheral plasma revealed that the majority of 5α-androstenone was present as a sulfoconjugate. The sulfoconjugated form of 5α-androstenone was found to be present up to 69±4.3% relative to its unconjugated form. The presence of this steroid in plasma was confirmed by GC-MS. The MO-TMS derivatives of the HPLC-purified steroids were identical to that of the authentic steroid standards. The GC retention time for the steroid standard of 5α-androstenone was 12.52, which corresponded to a peak at 12.56 min in the sulfoconjugate fraction. This peak was confirmed to be 5α-androstenone as it produced a molecular ion at m/z 301, with fragment ions at m/z 286 and m/z 270, which are identical to that of the 5α-androstenone standard (data not shown). GC-MS analysis also confirmed the presence of 3β-androstenol and 3α-androstenol as sulfoconjugates in peripheral plasma; however there was no detectible 16-androstenes present as glucuronide conjugates.

Analysis of testicular vein serum produced similar results to that of the peripheral plasma, with proportions of 5α-androstenone present up to 72±6.2% in the sulfoconjugate fraction relative to the unconjugated fraction. However, the overall concentration of 5α-androstenone in testicular vein serum was approximately 10 times greater than that found in the peripheral plasma, reaching concentrations of 350 ng/ml in some animals. The presence of this steroid in the sulfoconjugate fraction was confirmed by GC-MS, producing similar results to those reported above for peripheral plasma. As with peripheral plasma, 3β-androstenol and 3α-androstenol were present primarily as sulfoconjugates. Additionally, the 16-androstenes were not present within the glucuronide fraction of the testicular vein serum.

Relationships Between Steroid Concentrations

A negative correlation was observed (r=−0.36; P<0.01) between plasma concentrations of 5α-androstenone in the sulfoconjugate fraction and concentrations of 5α-androstenone in fat. Animals with fat androstenone concentrations less than 0.5 μg/g had plasma 5α-androstenone levels that ranged from 9 ng/ml to 56 ng/ml in the sulfoconjugate fraction (data not shown). However, high concentrations in the sulfoconjugate fraction were only present in low fat androstenone animals. This relationship is further characterized upon examining the unconjugated plasma fraction (data not shown). There was a significant positive correlation (r=0.31; P<0.01) between unconjugated 5α-androstenone in plasma and the concentrations of 5α-androstenone in fat. All of the animals with fat androstenone concentrations less than 0.5 μg/g had levels of 5α-androstenone below 9 ng/ml. High levels of unconjugated 5α-androstenone in the plasma were only associated with fat concentrations greater than 0.5 μg/g. Animals with high concentrations of unconjugated 5α-androstenone had corresponding low levels of sulfoconjugated 5α-androstenone, both of which related to increased concentrations in fat.

Inhibition Studies

The effects of various sulfotransferase inhibitors on the ability of the Leydig cells to produce sulfoconjugated 16-androstene steroids were tested. Triethylamine caused decreased production of sulfoconjugated 16-androstenes from pregnenolone in a dose-dependant manner, with concentrations reaching near zero levels at 100 μM triethylamine. Conversely, there was an increased production of the unconjugated forms of these steroids when the cells were treated with triethylamine. The viability of the Leydig cells treated with 100 μM of triethylamine was 85%.

Estrone had a very slight inhibitory effect on the production of sulfoconjugated 16-androstene steroids; however, this was only present at the 100 μM level. PCP had no effect on the production of sulfoconjugated 16-androstene steroids. After 8 h of exposure to 100 μM of PCP, a significant toxicity to Leydig cells was apparent, with viability decreasing to less than 50%.

Discussion

A large proportion of 5α-androstenone in the peripheral plasma is found in its sulfoconjugated form. However, 5α-androstenone does not contain any hydroxyl groups that would allow for sulfoconjugation. The 3-keto group of 5α-reduced steroids have been reported to undergo enolisation in many species (Drmanovic et al., 1999; Kouretas et al., 1996). Therefore, conjugation of 5α-androstenone likely occurs through an initial enolisation of the 3-keto group to a 3-enol form. The high level of sulfoconjugation of this steroid is in agreement with that found for many other steroids produced by the boar (Booth, 1983; Raeside and Howells, 1971). In fact, DHEA has been reported to be present primarily as a sulfoconjugate, reaching proportions of 90% relative to its unconjugated form (Tan and Raeside, 1980). The high concentration of sulfoconjugated 5α-androstenone present in testicular vein plasma indicates that the testes are of major importance in contributing to levels present in peripheral plasma; however, the hepatic contribution to these levels is unknown.

Metabolism of testicular steroid hormones into secondary products may alter their biological activity or advance their clearance from the body, thus affecting plasma concentrations. The process of sulfoconjugation has been classically thought to function as a mechanism to facilitate the metabolic clearance of the steroid; however, the high levels of sulfoconjugated steroids present in the plasma suggest that the biological significance of sulfoconjugation may be more complex. It has been suggested that the sulfoconjugates of testicular steroids may act as regulators of androgen and estrogen synthesis by controlling the levels of unconjugated steroids that are capable of interacting with their respective receptors (Payne and Jaffe, 1970; Raeside et al., 1999a).

In addition to the potential biological significance of sulfoconjugation, a drastic change in the physiochemical properties of the steroid occurs due to an increase in polarity and thus water-solubility (Bongiovanni and Cohn, 1970; Strott S. A., 1996). The findings of this study indicate that sulfoconjugation of 5α-androstenone limits the amount of the non-polar unconjugated form that is available to accumulate in adipose tissue. Animals with high concentrations of sulfoconjugated 5α-androstenone in plasma displayed low levels of 5α-androstenone in fat. In addition, these animals had relatively low levels of unconjugated 5α-androstenone in plasma, and therefore were unable to accumulate fat levels higher than 0.5 μg/g. The relationship between the level of androstenone in plasma and fat has been investigated in a number of studies, with contradictory results, ranging from no correlation (Bonneau et al., 1982; Lundstrom et al., 1978; Malmfors and Andresen, 1975) to positive correlations (Andresen, 1976; Groth and Claus, 1977; Sinclair et al., 2001a). These contradictions are likely the result of confounding factors such as physiological maturity as well as the genetic capacity to produce and metabolize the 16-androstene steroids. The results of the present study indicated that the extent to which 5α-androstenone accumulates in fat is ultimately related to the level of unconjugated steroid present in plasma. These findings suggested that the concentration of unconjugated 5α-androstenone in plasma is the result of a balance between the capacities for testicular 16-androstene synthesis and subsequent sulfoconjugation.

The ability to produce high levels of sulfoconjugated steroids depends on the levels and enzyme activities of the testicular sulfotransferases. EST and PST displayed very little to no action towards the 16-androstene steroids respectively, since both EST and PST are specific for hydroxyl groups on phenolic steroids. EST has been demonstrated to be capable of sulfating hydroxysteroids; however this sulfation is relatively inefficient (Negishi et al., 2001). The results of the inhibition studies indicate that the specific sulfotransferase responsible for conjugating the 16-androstene steroids is HST. HST that is expressed in the testicular tissue of the boar has been shown to localize in the Leydig cells and has a broad substrate specificity (Hobkirk et al., 1989). HST prefers steroid substrates with 3β-hydroxy acceptor sites; however, HST is capable of acting on steroids with 3α and 17β-hydroxy acceptor sites (Falany et al., 1989; Pedersen et al., 2000; Strott S. A., 1996). Therefore, the 3β- and 3α-hydroxyl groups of 3β-androstenol and 3α-androstenol respectively, serve as potential acceptor sites for sulfoconjugation by HST. It is also likely that the hydroxyl group at the 3 position of the proposed enol form of 5α-androstenone is sulfoconjugated by HST.

A significant negative correlation was determined between plasma concentrations of 5α-androstenone present in the sulfate fraction and the concentrations of 5α-androstenone present in fat. This suggested that the accumulation of 5α-androstenone in fat is influenced by the proportion of the sulfoconjugated form present in the peripheral plasma. HST was found to be the key enzyme involved in the sulfoconjugation of the 16-androstene steroids and can play a significant role in determining the levels of sulfated steroids present in plasma.

### Example 2

### Molecular Cloning and Expression of the Porcine SULT2A1 Gene: Its Functional Relevance to Boar Taint

Sulfotransferase enzymes are cytosolic proteins that are involved in catalyzing the conjugation of many steroids, bile acids, and xenobiotics. Sulfotransferases utilize the donor molecule 3′-phosphoadenosine 5′phosphosulfate (PAPS) for the transfer of a sulfate radical (SO₃₋) to a hydroxyl acceptor site (Robbins, 1956). In terms of steroids, hydroxyl groups at positions 3, 21, and 17 of the steroid nucleus are the most common locations for sulfoconjugation (Strott S. A., 1996). With the addition of the sulfate group, the polarity of the steroid conjugate greatly increases, causing an increase in water solubility (Bongiovanni and Cohn, 1970; Jakoby et al., 1980). Therefore, sulfoconjugation of hydroxysteroids has been regarded as a major mechanism for their metabolism and excretion (Mulder, 1981).

Steroid sulfotransferase enzymes are located in the liver and other organs such as the adrenal glands, ovary, and testis (Gasparini et al., 1976; Hobkirk, 1985; Roberts and Lieberman, 1970). In the boar, a main organ responsible for steroid sulfate synthesis is the testes (Hobkirk et al., 1989; Raeside and Renaud, 1983). One of the major steroid sulfotransferases is hydroxysteroid sulfotransferase. Hydroxysteroid sulfotransferase has a very large substrate specificity; however, its primary substrate is dehydroepiandrosterone (DHEA) and has thus been named DHEA-sulfotransferase in the past (Comer et al., 1993; Falany et al., 1989). In recent years DHEA-sulfotransferase has been further classified to belong to the 2A family of human sulftotransferases, and is has been designated as SULT2A1 by the HUGO Nomenclature Committee.

A hydroxysteroid sulfotransferase has recently been reported to be responsible for sulfoconjugating the 16-androstene steroids (see Example 1). The 16-androstene steroids are the most quantitatively abundant steroids produced by the boar testes, reaching total levels of approximately 0.6 mg/g of testicular tissue (Booth, 1975; Booth and Polge, 1976; Gower, 1972). The 16-androstenes are known for their involvement in boar taint (Gower, 1972; Patterson, 1968). Boar taint is partly due to the accumulation of high levels of 5α-androst-16-en-3-one (5α-androstenone) in adipose tissue, which produces an unpleasant odor upon heating or cooking of the fat. Previous studies indicated that increased levels of sulfoconjugated 16-androstene steroids present in the systemic circulation are associated with a reduction in the accumulation of 5α-androstenone in adipose tissue (see Example 1). However, it is unknown whether the presence of sulfoconjugated 16-androstenes in the circulation is regulated by the activity of porcine SULT2A1 in various tissues.

In humans, SULT2A1 activities have been reported to vary among individuals up to 5-fold, with individuals belonging to either low or high activity subgroups (Aksoy et al., 1993; Weinshilboum and Aksoy, 1994). These findings suggested that genetic polymorphisms can be involved in regulating enzyme activity. Single nucleotide polymorphisms (SNPs) within the human SULT2A1 gene have been observed in a number of studies (Igaz et al., 2002; lida et al., 2001; Ottemess et al., 1995a), some of which have resulted in reductions in the levels of both enzyme activity and the level of protein (Thomae et al., 2002; Wood et al., 1996).

Because of the significant role of sulfoconjugation of the 16-androstene steroids in the development of boar taint, it was important to understand the molecular basis for individual variation in the expression and function of the SULT2A1 gene in market weight boars. Therefore, the determination of the porcine SULT2A1 cDNA sequence was necessary. In addition, genetic polymorphisms in the SULT2A1 gene which cause alterations in enzyme function were examined. Investigation into how porcine SULT2A1 genetic variation translates into interindividual differences in 5α-androstenone accumulation in fat is of great importance in identifying a potential candidate gene and developing genetic markers for boar taint.

Materials and Methods

Tissue Samples

A total of 28 Yorkshire boars of 175±6 days of age were obtained from the Arkell Swine Research Station at the University of Guelph. Blood samples were taken from the orbital sinus, centrifuged at 4° C. to collect plasma and stored at −20° C. until extraction and analysis for 5α-androstenone concentrations.

The animals were slaughtered at an average live weight of 125×13 kg. A backfat sample was removed from the midline on the point of the 11^{th} rib and frozen at −20° C. until assayed for 5α-androstenone. Samples of liver and testis tissue were taken immediately following exsanguination, frozen in liquid nitrogen and stored at −70° C. before use.

Steroid Extraction and Analysis

Prior to analysis of blood samples, conjugated steroids were separated from unconjugated steroids with the use of methanol primed Sep-Pak C₁₈ solid-phase chromatography cartridges (Raeside and Christie, 1997). Sulfoconjugated steroids were hydrolyzed by incubating the conjugate fraction overnight in trifluoroacetic acid/ethyl acetate (1/100 v/v) at 45° C. The hydrolyzed steroids were then purified by Sep-pak C₁₈ solid-phase chromatography.

Fat and extracted plasma samples were analyzed for 5α-androstenone with an ELISA method, modified after (Claus et al., 1988) as described previously (Squires and Lundstrom, 1997).

Preparation of Cytosol

Frozen testes and liver samples were partially thawed and a 20% (w/v) homogenate was prepared in 100 mM Tris-HCL, 10 uM EDTA, 250 mM sucrose, pH 7.4. Cytosolic fractions were obtained by differential centrifugation. Homogenates were centrifuged for 15 min at 10000×g at 4° C. and the resulting supernatant was removed and centrifuged for 60 min at 105000×g at 4° C. The supernatant (cytosol) was removed and stored at −70° C. The protein content of the cytosol preparations was estimated using the Bio-Rad protein assay, based on the Bradford method using bovine serum albumin as the standard.

Sulfotransferase Activity Assay

Sulfotransferase reactions were assayed by the method previously described (Matsui et al., 1993) using DHEA as a substrate. The incubation media contained 100 mM Tris/HCL, 100 uM EDTA, 100 uM PAPS, 10 mM MgCl₂ and 50 uM [³H] DHEA (approx 0.01-0.05 uCi/nmol) in a final volume of 500 ul at pH 7.4. Incubations were carried out at 37° C. for 15 minutes with 25 μg of total cytosolic protein. Blank values were obtained with incubations that lacked PAPS. The reactions were terminated with the addition of 100 ul of 0.1N NaOH, followed by snap freezing with liquid nitrogen. The samples were then immediately extracted by C₁₈ solid-phase chromatography, as described above. After sep-pak extraction, 100 ul aliquots of the unconjugated and conjugated fractions were subjected to liquid scintillation counting.

Screening of a Porcine cDNA RACE Library and Sequence Analysis

5′ and 3′ rapid amplification of cDNAs (RACE) were constructed from 1 μg of total RNA from liver with the use of the Smart RACE cDNA amplification kit (BD Biosciences) as described previously (Lin et al., 2004). The cDNA library was used as a template in the subsequent PCR screening of porcine SULT2A1. The first fragment of porcine SULT2A1 was amplified with the primers designed from a porcine expressed sequence tag (Accession #: BI402591) related to reproductive function that was 82% homologous to human SULT2A1. To obtain the full-length porcine SULT2A1 cDNA, forward and reverse primers were designed based on the sequence obtained from the 5′ and 3′ RACE and used to amplify the full-length porcine SULT2A1 with either 5′ or 3′ RACE cDNA as a template. The nucleotide sequence of the forward primer was 5′ CACGAGGCGCAAAGAACT 3′ (SEQ ID NO:4), whereas the reverse primer was 5′ CATGTGCAAGGACAGGTGAG 3′(SEQ ID NO:5). The PCR consisted of 35 cycles of denaturing for 1 minute at 94° C., annealing for 1 minute at 63° C., and extending for 1 minute at 72° C. A final extension step was performed for 10 minutes at 72° C. Ten microlitres of the PCR product was analyzed by electrophoresis on a 1% agarose gel.

The PCR fragments were ligated into pGEM-T Easy Vector System (Promega), and then transformed into competent DH5α cells. Plasmid DNAs were purified and subjected to sequence analysis using an ABI 377 DNA sequencer (Applied Biosystems).

Isolation of Total RNA and Reverse Transcription-PCR

100 mg of testes and liver tissue were homogenized in 1 ml of Tri-Reagent (Sigma) and incubated at room temperature for 10 min. Following incubation, 0.2 ml of chloroform was added followed by vortexing and centrifugation at 12000×g for 10 min at 4° C. The supernatant was mixed with 0.5 ml of isopropanol and incubated at room temperature for 10 min. The RNA was collected by centrifugation for 10 min and the RNA pellet was washed with 75% ethanol and re-suspended in 50 μl of DEPC treated water.

Approximately 0.5 μg of total RNA was used to synthesize first strand cDNA using Superscript II RNase H⁻ Reverse Transcriptase (Invitrogen Life Technologies, Carlsbad, Calif.), following the manufacturers instructions. The RT reaction was performed at 25° C. for 10 min, 42° C. for 50 min, followed by a 15 min incubation at 70° C. First-strand cDNAs were stored at 4° C. until SSCP or RT-PCR analysis.

Following the reverse transcription reaction, 2.5 μl of the first strand cDNA was used as a template for PCR. The PCR mixture contained 100 mM Tris/HCL pH 8.3, 500 mM KCl, 11 mM MgCl₂, 0.1% gelatin, 0.2 mM dNTP, 2.5 U of Red Taq polymerase (Sigma), and 0.4 mM of the porcine SULT2A1 forward and reverse primers. The PCR profile was the same as stated above.

Single-Strand Conformational Polymorphism (SSCP) Analysis

The PCR products from both testicular and hepatic cDNAs were digested into fragments (230, 345 and 459 bp) with XmiI and SacI restriction endonucleases (Fermentas) for 3 hours at 37° C. A total of 7 μl of the digested cDNA fragments were combined with 13 μl of loading buffer (10% sucrose, 0.01% Bromophenol blue, 0.01% Xylene cyanol FF). The samples were then denatured at 100° C. for 5 min, followed by immediate cooling on ice. The samples were then loaded onto a 10% polyacrylamide gel. Electrophoresis was carried out for 17 h at 160 V using a 130×160×1 mm vertical unit (Bio-Rad Laboratories) connected to a controlled refrigerated circulator maintained at 15° C. After electrophoresis the gels were silver stained to resolve the individual banding patterns. Polymorphisms were verified by sequencing all of the samples.

Western Blot Analysis

Testicular and hepatic total cytosolic protein concentrations were adjusted to 15 μg/μl and 50 μg/μl respectively in a final volume of 20 ul with the addition of loading buffer (0.5 M Tris-HCI pH 6.8, 45% glycerol, 0.5 M EDTA, 10% SDS, 0.05% bromophenol blue, and 10 mM β-mercaptoethanol). Samples were boiled for 2 min, and 10 μl of sample was resolved on 12% polyacrylamide gels. Electrophoresis was carried out for approximately 3 hours at 75 V, followed by electrophoretic transfer to Hybond-C nitrocellulose membranes (Amersham Canada) at 35 V. Immunoreactive porcine SULT2A1 protein was measured by using a commercially available polyclonal antibody to human SULT2A1 (MBL) used at a 1:5000 dilution. The secondary antibody was a 1:2000 dilution of donkey anti-rabbit IgG horseradish peroxidase (Amersham Canada). Blots were visualized by chemiluminescence detection and subsequently quantified using a densitometer.

Real-Time PCR

Real Time PCR amplification was performed using a Copeheid Smart Cycler System. Forward and reverse primers were designed based on the porcine SULT2A1 sequence. The forward primer 5′ CCATGCGAGACAAGGAGAAC 3′ (SEQ ID NO:6), and reverse primer 5′ CATGACCTGGAAGGAGCTGT 3′ (SEQ ID NO:7) amplified a product of 155 bp in length at annealing temperature of 70° C. The QuantiTect SYBR Green PCR Amplification (Qiagen) kit was used for the real-time quantification of the PCR products. The real-time PCR reaction consisted of 2.5 μl cDNA, 20 μM of the forward and reverse primers, in a total volume of 25 μl. As an internal control, the hypoxanthine-guanine phosphoribosyltransferase (HPRT) gene was amplified for each real-time PCR reaction. HPRT amplification included the forward primer 5′ CTTTGCTGACCTGCTGGATT 3′ (SEQ ID NO:8), and reverse primer 5′ CTTGACCAAGGAAAGCAAGG 3′ (SEQ ID NO:9), which amplified a product of 232 bp in length. Relative quantification of mRNA levels between animals with different levels of SULT2A1 protein were analyzed by the 2(-Delta Delta C(T)) method (Livak and Schmittgen, 2001).

Statistical Analyses

Pearson correlation coefficients were calculated for the following measures: 1) SULT2A1 activity vs. plasma concentrations of sulfoconjugated 5α-androstenone 2) SULT2A1 activity vs. fat concentrations of 5α-androstenone (SAS 8.2, SAS Inst. Inc.). Correlations were considered statistically significant at if p values were less or equal to 0.05. Differences in SULT2A1 activity and protein levels between high and low boar taint pigs were analyzed by t-tests (SAS 8.2, SAS Inst. Inc.).

Results

Relationship between SULT2A1 Activity and 5α-androstenone

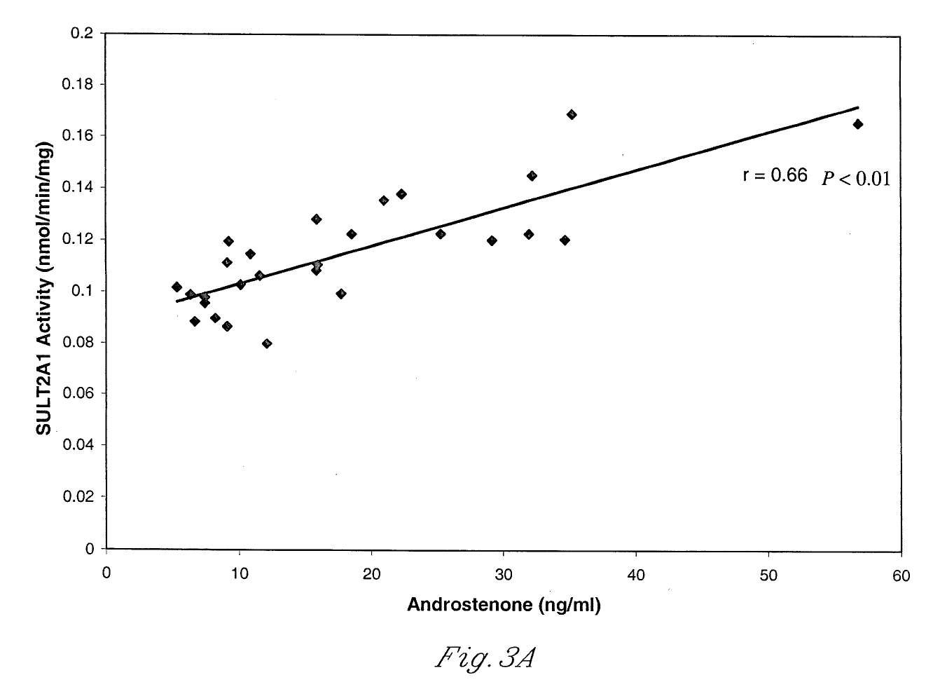

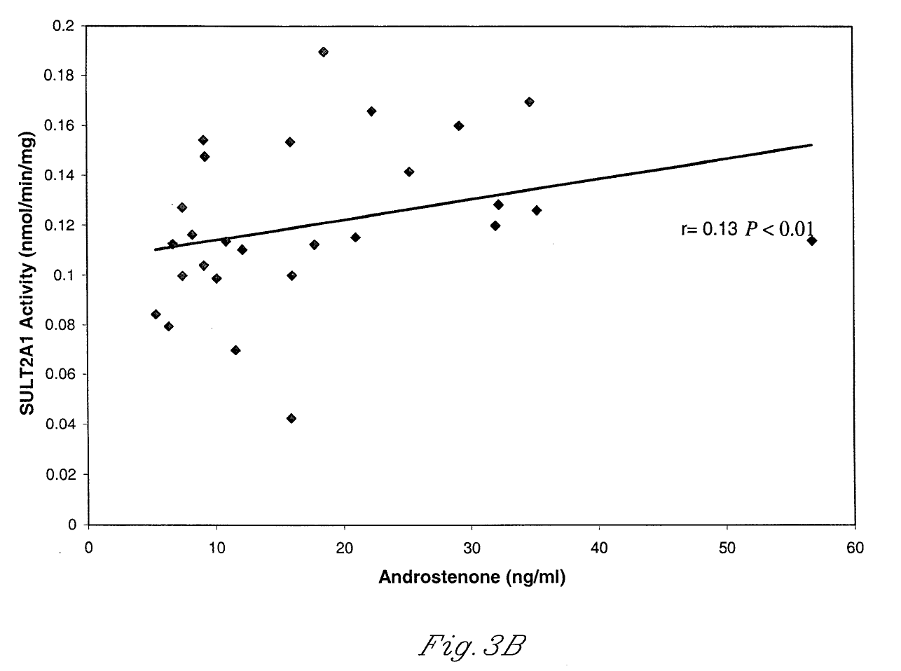

A strong positive correlation (r=0.66; P<0.01) was observed between the testicular activity of SULT2A1 and plasma concentrations of 5α-androstenone in the sulfoconjugate fraction (FIG. 3A); however this relationship was not as strong (r=0.13; P<0.01) with respect to hepatic SULT2A1 activity (FIG. 3B). In terms of testicular SULT2A1 activity, animals with the highest sulfotransferase activity had the highest concentrations of sulfated 5α-androstenone in peripheral plasma; levels were as high as 57 ng/ml. In addition, these animals also had the lowest concentrations of unconjugated 5α-androstenone, at levels below 9 ng/ml (data not shown).

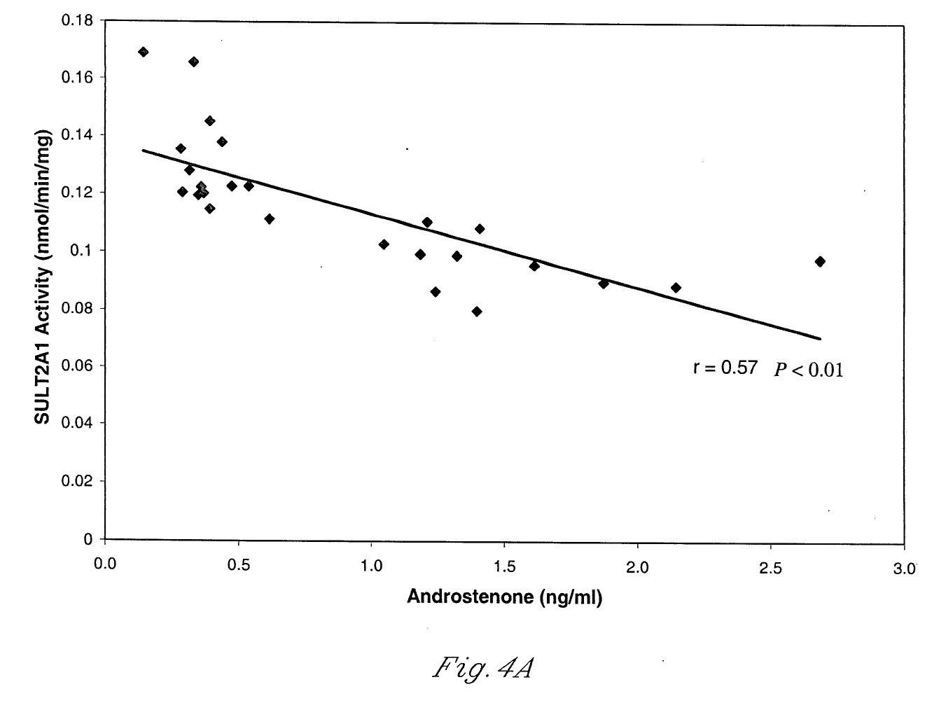

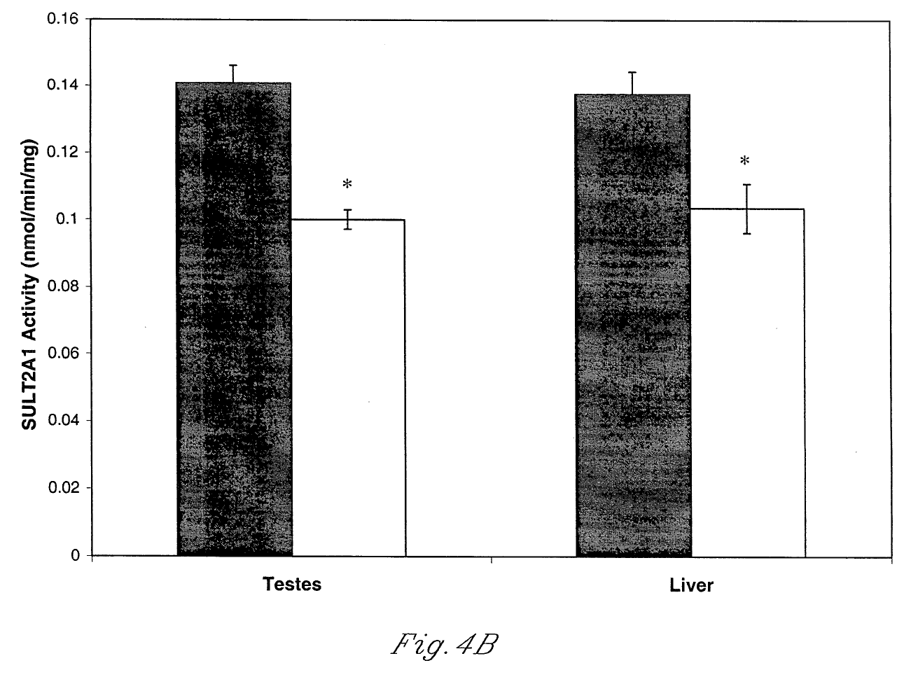

There was a statistically significant negative correlation (r=−0.57; P<0.01) between testicular SULT2A1 activity and the concentrations of 5α-androstenone in fat (FIG. 4A). All of the animals with fat 5α-androstenone concentrations less than 0.5 μg/g had corresponding high SULT2A1 activities. This relationship was also observed to a lesser extent (r=−0.31; P<0.05) for hepatic SULT2A1 activity (data not shown).

In order to determine the relationship between SULT2A1 activity and 5α-androstenone accumulation in fat, animals were separated into two groups based on fat 5α-androstenone concentrations either above or below the limit of 0.5 μg/g. Both testicular and hepatic SULT2A1 activities (FIG. 4B) were significantly lower in animals with fat 5α-androstenone concentrations higher than 0.5 μg/g.

Isolation and Sequence Characterization of Porcine SULT2A1 cDNA

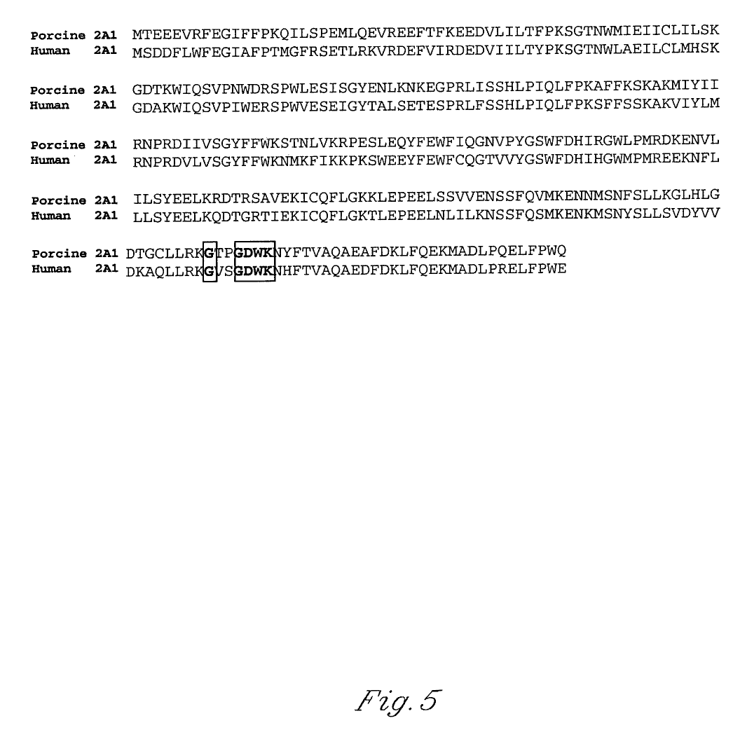

The nucleotide sequence of the porcine SULT2A1 cDNA was 1036 bp long and contained an 858 bp-long open reading frame (ORF), which encodes for 285 amino acids (FIG. 2). The porcine SULT2A1 ORF displayed 82% homology to human SULT2A1, 84% homology to mouse hydroxysteroid sulfotransferase, and 85% homology to rat hydroxysteroid sulfotransferase. The deduced amino acid sequence for porcine SULT2A1 displayed 81% homology to human SULT2A1, 80% homology to mouse hydroxysteroid sulfotransferase, and 80% homology to rat hydroxysteroid sulfotransferase. In humans, Gly248, Val249, Ser250, Gly251, Asp252, Trp253, and Lys254 are reported to be the active site for SULT2A1, specifically acting as a PAPS binding site. In the porcine sequence, Gly248, Gly251, Asp252, Trp253, and Lys254 are conserved (FIG. 5).

SULT2A1 Genetic Polymorphisms

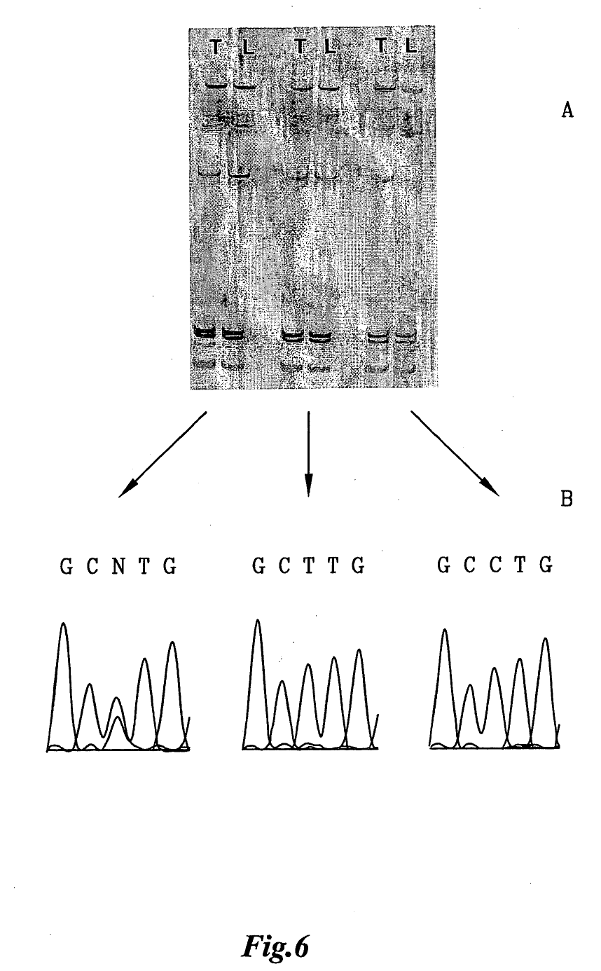

SSCP was used to scan for genetic polymorphisms in the porcine SULT2A1 coding region from both testicular and liver samples. Three different types of banding patterns were detected (FIG. 6). Sequence analysis showed that the observed SSCP banding patterns corresponded to a mutation from a cytosine to a thymine within the coding region at bp position 219. In fact, all animals could be genotyped as being homozygous for either of these conditions or heterozygous in which a cytosine and thymine were present. This mutation did not cause a change in amino acid sequence and is therefore a silent mutation that is not likely associated with SULT2A1 activity.

Western Blot Analysis

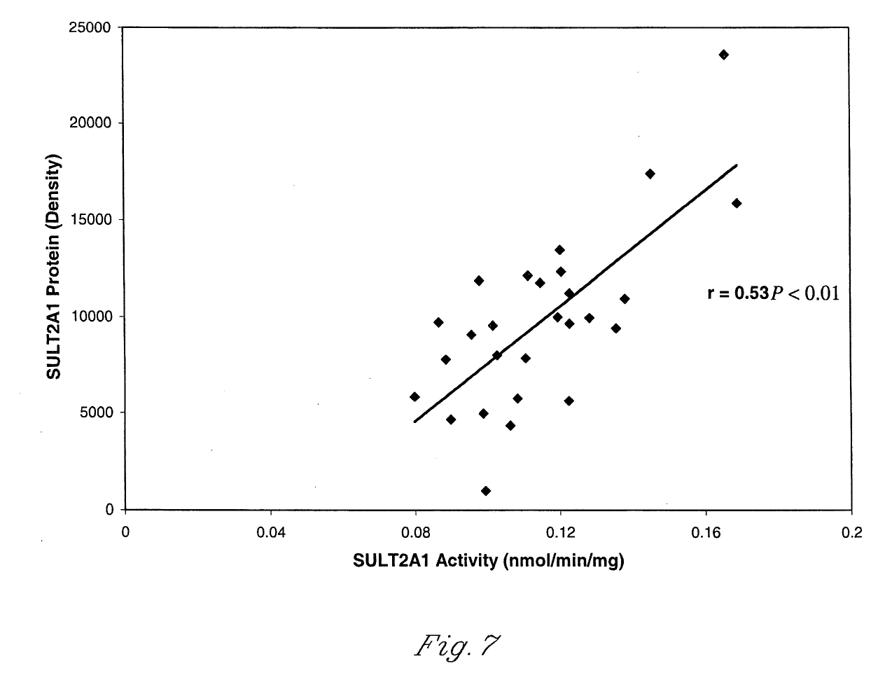

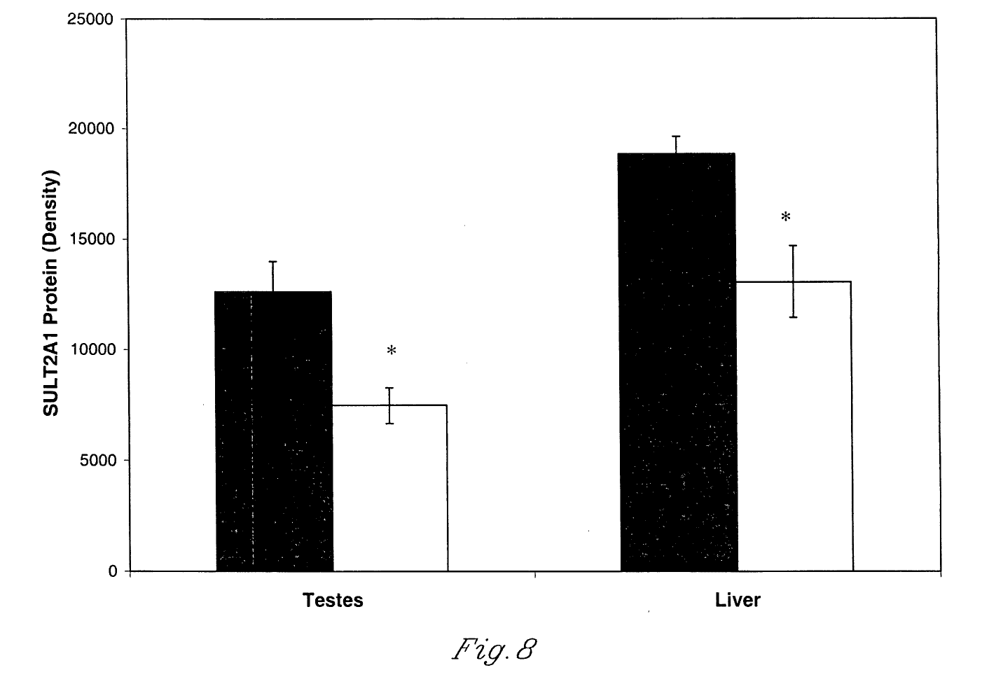

The lack of functional polymorphisms within the coding region warranted investigation into whether the observed variation in SULT2A1 activity was due to individual differences in the amount of SULT2A1 protein that is produced. Following immunoblot analysis, it was determined that the differences in SULT2A1 activity were due to differences in the level of SULT2A1 protein. Animals with low SULT2A1 activity had low levels of both testicular and hepatic SULT2A1 protein, at times reaching 6 times less protein than animals with high SULT2A1 activity. FIG. 7 demonstrates the relationship between the testicular level of SULT2A1 protein and enzyme activity. A statistically significant positive correlation (r=0.53; P<0.01) between these two parameters was apparent with decreased levels of protein associated with decreased activity. A similar relationship was observed with hepatic levels of SULT2A1 (data not shown); however, the relationship was not as strong (r=0.22; P<0.05). In addition, there was a statistically significant positive correlation (r=0.42; P<0.01) between testicular SULT2A1 protein and the concentrations of 5α-androstenone in the sulfoconjugate fraction of the plasma. In terms of the relationship between hepatic SULT2A1 protein and 5α-androstenone in the sulfoconjugate fraction, the relationship was not as strong (r=0.14; P<0.01).

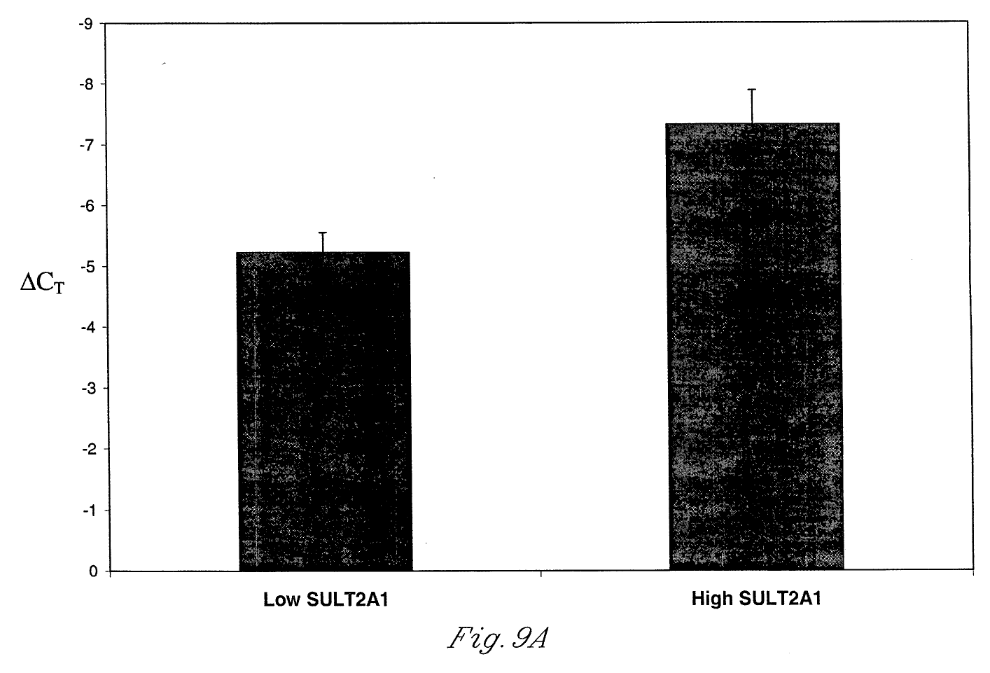

In terms of the relationship between the levels of testicular and hepatic SULT2A1 protein and 5α-androstenone accumulation in fat, the results were similar to that observed for SULT2A1 activity; animals with high levels of 5α-androstenone in fat had significantly lower levels of total SULT2A1 protein (FIG. 8).

Real Time PCR

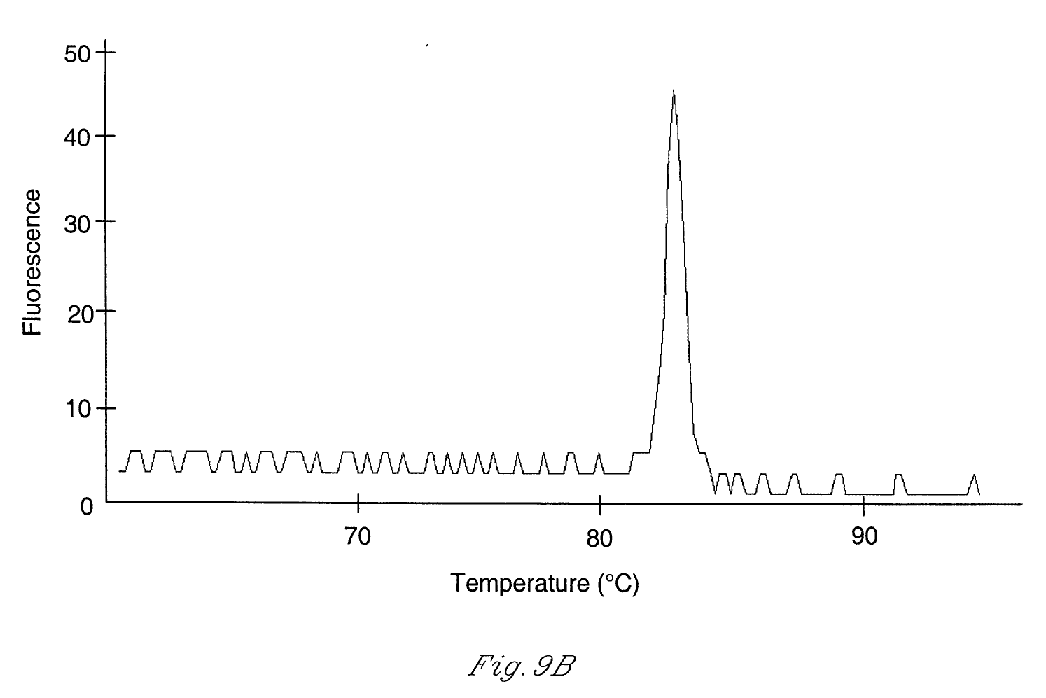

In order to determine whether the difference in SULT2A1 protein expression is regulated at the transcriptional level, quantitative real-time PCR was performed to measure the levels of SULT2A1 mRNA. Animals with high levels of SULT2A1 protein in both testes and liver had a significantly higher (P<0.01) level of SULT2A1 mRNA than animals with low levels of the SULT2A1 protein (FIG. 9). Relative quantification of these results using the comparative 2(-Delta Delta C(T)) method revealed that the expression of the SULT2A1 gene was increased 3.5 fold in animals with high levels of the SULT2A1 protein relative to animals with low levels of the protein.

Discussion

The extent to which 5α-androstenone accumulates in fat is influenced by the amount of unconjugated steroid that is present in the circulation. Differences in the ability to sulfoconjugate 5α-androstenone will limit the level of unconjugated steroid that is available to accumulate in fat. The levels of sulfoconjugated 16-androstene steroids present in the circulation are a result of the balance between the capacity for testicular steroidogenesis, sulfoconjugation, and metabolic clearance. The results of this study show that the concentration of sulfoconjugated 5α-androstenone in the peripheral plasma is highly dependant on testicular SULT2A1 activity (r=0.66). However, in terms of hepatic SULT2A1 activity, this relationship was not as strong (r=0.13). This further clarifies the magnitude of testicular sulfoconjugation in the boar.

A high level of steroid sulfotransferase enzymes are present in the boar testis which are responsible for the large proportion of sulfoconjugated steroids that are secreted from this organ (Raeside et al., 1989; Raeside and Renaud, 1983; Tan and Raeside, 1980). The liver has also been found to be capable of sulfoconjugating the 16-androstene steroids, but to a lesser extent than the testis. Upon examination of SULT2A1 activity in high and low boar taint animals, it was found that animals with high concentrations of 5α-androstenone in fat had significantly lower SULT2A1 activity in both testis and liver tissues. These findings indicate that a low level of SULT2A1 activity results in decreased sulfoconjugation and thus, more of the unconjugated form of the steroid will be available to deposit into the adipose tissue of high boar taint pigs.

The relationship between SULT2A1 activity and the accumulation of 5α-androstenone in fat warranted investigation at the molecular level, as genetic polymorphisms within the SULT2A1 gene may account for the differences observed in enzyme activity. In order to test this hypothesis, the cDNA sequence for porcine SULT2A1 was required. Primers were designed based on a porcine expressed sequence tag that was highly homologous to human SULT2A1. Using a porcine cDNA library and the designed primers, it was possible to amplify the full length coding sequence of the porcine SULT2A1 cDNA. Porcine SULT2A1 was found to be highly homologous in its cDNA and amino acid sequences to its orthologous human, mouse and rat genes. These findings lead to the conclusion that the putative SULT2A1 is in fact porcine SULT2A1.

Human SULT2A1 has been mapped to chromosome 19q13.3 (Luu-The et al., 1995; Ottemess et al., 1995b), which is homologous with porcine chromosome 6q11, 21 and chromosome 7q12. The homology to porcine chromosome 7 is of interest, as many quantitiative trait loci (QTL) for phenotypic traits, including fat 5α-androstenone concentration, have been mapped to chromosome 7 (Quintanilla et al., 2003; Tanaka et al., 2003).

SULT2A1 is highly involved in the biotransformation of many steroid hormones, neurotransmitters, drugs and xenobiotics. Therefore, identification of genetic differences that cause changes in enzyme activity may be crucial in determining the individual response to specific compounds. In humans, substantial efforts have been made to detect genetic polymorphisms in SULT2A1 due to its involvement in cardiovascular disease and cancer (LaCroix et al., 1992; Shibutani et al., 1998; Stahl et al., 1992). Polymorphisms within the human SULT2A1 gene have been observed in a number of studies (Igaz et al., 2002; lida et al., 2001; Otterness et al., 1995a), some of which have resulted in reductions in the levels of both enzyme activity and the level of protein (Thomae et al., 2002; Wood et al., 1996). However, despite the fact that this enzyme is highly polymorphic, the mutation may not be functionally correlated to the activity or level of the protein (Aksoy et al., 1993). As disclosed herein, there were no significant functional polymorphisms detected within the ORF of the porcine SULT2A1 gene. Therefore, differences in enzyme activity can not be attributed to mutations within the coding region. There are multiple mechanisms that could alter the level of enzyme activity other than polymorphisms within the ORF of the cDNA. One major influence of enzymatic activity is the amount of functional protein within the tissue. After western analysis, it was determined that the differences observed in SULT2A1 activity were a result of the amount of SULT2A1 protein that was expressed within the tissue. Animals with low SULT2A1 activity in testis and liver tissues had correspondingly low levels of protein. This decrease in protein will therefore contribute to a decreased sulfation activity towards the 16-androstene steroids.

There are multiple regulatory mechanisms that are involved in controlling gene expression, many of which influence the level of functional protein that is produced. These regulatory processes can occur at multiple levels. At the transcriptional level, alterations in receptor-dependant mechanisms or transcription factors could lead to differences in the amount of protein that is transcribed (Tsai and O'Malley, 1994a). Post-transcriptional mechanisms such as mRNA stabilization can also play a significant role in determining the level of production of an end product (Day and Tuite, 1998). In addition, there are multiple processing events that operate at the translational and post-translational levels that could have an equal impact on the amount or stability of functional product. The results from the quantitative real-time PCR analysis suggest that level of SULT2A1 production is regulated at the transcriptional level, as animals with high levels of SULT2A1 expressed 3.5 fold more mRNA for SULT2A1 than animals with low levels of the protein.

The molecular mechanisms that control SULT2A1 gene transcription have not been fully characterized. Chenodeoxycholic acid has been demonstrated to be a strong inducer of the rat SULT2A1 gene (Makishima et al., 1999). This inducing effect is controlled by the bile acid-activated famesoid X receptor (FXR) (Song et al., 2001). The FXR will subsequently form a heterodimer with the retinoid X receptor (RXR) in order to interact with a defined FXR/RXR response element to promote transcription. Similarly, human SULT2A1 has been shown to be induced in response to dexamethasone (Duanmu et al., 2002). The response to dexamethasone suggests that the pregnane X receptor (PXR) transcription factor has been activated, which can be activated by many steroidal ligands such as pregnenolone (Kliewer et al., 1998). More recently, it has been determined that the constitutive androstane receptor (CAR) is also potentially involved in SULT2A1 regulation (Saini et al., 2004). Therefore, differences in the level of gene transcription can be due to individual differences in the proximal promoter region of the gene where the recognition sequences for these transcription factors are located.

In summary, the accumulation of 5α-androstenone in fat is influenced by the individual capacity for sulfoconjugation. SULT2A1 has been identified to be the key enzyme involved in the sulfoconjugation of the 16-androstene steroids. After isolation of the porcine SULT2A1 cDNA, it was determined that differences in enzyme activity were not the result of genetic polymorphisms in the coding region. The proportion of sulfoconjugated 5α-androstenone present in the circulation is highly dependant on the level of SULT2A1 protein that is expressed and thus, its relative activity. The finding that animals with low levels of SULT2A1 have resulting higher levels of 5α-androstenone concentrations in fat suggests that sulfoconjugation plays a vital role in regulating the level of unconjugated or “free” steroid that is capable of accumulating in adipose tissue. Based on these results, SULT2A1 could potentially be used as a genetic marker for selecting animals with low boar taint.

### REFERENCES

Aksoy, I. A., V. Sochorova, and R. M. Weinshilboum. 1993. Human liver dehydroepiandrosterone sulfotransferase: nature and extent of individual variation. Clin. Pharmacol. Ther. 54:498-506.
Allrich, R. D., R. K. Christenson, J. J. Ford, and D. R. Zimmerman. 1982. Pubertal development of the boar: testosterone, estradiol-17 beta, cortisol and LH concentrations before and after castration at various ages. J. Anim Sci 55:1139-1146.
Andresen, O. 1976. Concentrations of fat and plasma 5alpha-androstenone and plasma testosterone in boars selected for rate of body weight gain and thickness of back fat during growth, sexual maturation and after mating. J. Reprod. Fertil. 48:51-59.
Andresen, O., T. Froystein, M. Rodbotten, H. P. Mortensen, O. Eik-Nes, and P. Lea. 1993. Sensoric evaluation of boar meat with different levels of androstenone and skatole. In: M. Bonneau (Ed.) Measurement and Prevention of boar taint in entire male pigs. pp. 69. INRA Editions, Paris.
Babol, J. and E. J. Squires. 1995. Quality of Meat from Entire Male Pigs. Food Research International 28:201-212.
Babol, J., E. J. Squires, and M. Bonneau. 1996. Factors regulating the concentrations of 16-androstene steroids in submaxillary salivary glands of pigs. J. Anim Sci 74:413-419.
Babol, J., E. J. Squires, and K. Lundstrom. 1999. Relationship between metabolism of androstenone and skatole in intact male pigs. J. Anim Sci. 77:84-92.
Baranczyk-Kuzma, A. and A. Ciszewska-Pilczynska. 1989. Sulfation in male reproductive organs. Bull and boar testis phenol sulfotransferases. Biochem. Pharmacol. 38:4231-4236.
Bejerholm, A. C. and P. A. Barton-Gade. 1993. The relationship between skatole/androstenone content and odour/flavour of meat from entire male pigs. In: M. Bonneau (Ed.) Measurement and Prevention of Boar Taint in Entire Male Pigs. pp. 75. INRA Editions, Paris.
Boles, J. W. and C. D. Klaassen. 1998. Effects of molybdate and pentachlorophenol on the sulfation of dehydroepiandrosterone. Toxicol. Appl. Pharmacol. 151:105-109.
Bongiovanni, A. M. and R. M. Cohn. 1970. Clinical Aspects of Steroid Conjugation. In: S. Bernstein and S. Solomon (Eds.) Chemical and Biological Aspects of Steroid Conjugation. pp. 409-456. Springer-Verlag, New York.
Bonneau, M. 1987. Effects of age and live weight on fat 5 alpha-androstenone levels in young boars fed two planes of nutrition. Reprod. Nutr. Dev. 27:413-422.
Bonneau, M., N. Meussy-Dessolle, P. C. Leglise, and R. Claus. 1982. Relationships between fat and plasma androstenone and plasma testosterone in fatty and lean young boars following castration. Acta Endocrinol. 101:129-133.
Bonneau, M. and P. Sellier. 1986. Fat androstenone content and development of the genital system in young Large White boars: genetic aspects. World. Rev. Anim. Prod. 22:27-34.
Booth, W. D. 1975. Changes with age in the occurrence of C19 steroids in the testis and submaxillary gland of the boar. J. Reprod. Fertil. 42:459-472.
Booth, W. D. 1983. Development of some male characteristics supported by oestrone but not dehydroepiandrosterone in the boar. J. Reprod. Fertil. 68:9-16.
Booth, W. D. and C. Polge. 1976. The occurrence of C 19 steroids in testicular tissue and submaxillary glands of intersex pigs in relation to morphological characteristics. J. Reprod. Fertil. 46:115-121.
Claus, R., G. Mahler, and E. Munster. 1988. Determination of boar taint steroid 5α-androst-16-en-3-one in adipose tissue of pigs with a rapid microtiter plate enzyme-immunoassay (MTE). Archiv. Lebensmittelhyg 39:87-96.
Claus, R., U. Weiler, and A. Herzog. 1994. Physiological-Aspects of Androstenone and Skatole Formation in the Boar—A Review with Experimental-Data. Meat Science 38:289-305.
Comer, K. A., J. L. Falany, and C. N. Falany. 1993. Cloning and expression of human liver dehydroepiandrosterone sulphotransferase. Biochem. J. 289 (Pt 1):233-240.
Davis, S. M. and E. J. Squires. 1999. Association of cytochrome b5 with 16-androstene steroid synthesis in the testis and accumulation in the fat of male pigs. J. Anim Sci. 77:1230-1235.
Day, D. A. and M. F. Tuite. 1998. Post-transcriptional gene regulatory mechanisms in eukaryotes: an overview. J. Endocrinol. 157:361-371.
deLange, C. F. M. and E. J. Squires. Entire males vs castrates for pork production—financial benefits to the producer. 0842-9839, 44. 1995. Universitiy of Guelph, ON, Canada, OAC publication. Ontario Swine Research Review.
Drmanovic, Z., S. Voyatzi, D. Kouretas, D. Sahpazidou, A. Papageorgiou, and O. Antonoglou. 1999. Albumin possesses intrinsic enolase activity towards dihydrotestosterone which can differentiate benign from malignant breast tumors. Anticancer Res. 19:4113-4124.
Duanmu, Z., D. Locke, J. Smigelski, W. Wu, M. S. Dahn, C. N. Falany, T. A. Kocarek, and M. Runge-Morris. 2002. Effects of dexamethasone on aryl (SULT1A1)- and hydroxysteroid (SULT2A1)-sulfotransferase gene expression in primary cultured human hepatocytes. Drug Metab Dispos. 30:997-1004.
Falany, C. N. 1997. Enzymology of human cytosolic sulfotransferases. FASEB J. 11:206-216.
Falany, C. N., M. E. Vazquez, and J. M. Kalb. 1989. Purification and characterization of human liver dehydroepiandrosterone sulphotransferase. Biochem. J. 260:641-646.
Falany, C. N., J. Wheeler, T. S. Oh, and J. L. Falany. 1994. Steroid sulfation by expressed human cytosolic sulfotransferases. J. Steroid Biochem. Mol. Biol. 48:369-375.
Fayz, S., W. F. Cherry, J. R. Dawson, G. J. Mulder, and K. S. Pang. 1984. Inhibition of acetaminophen sulfation by 2,6-dichloro-4-nitrophenol in the perfused rat liver preparation. Lack of a compensatory increase of glucuronidation. Drug Metab Dispos. 12:323-329.
Fortin, A., W. D. Friend, and N. K. Sarkar. 1983. A note on the carcass composition of Yorkshire boars and barrows. Can. J. Anim. Sci 63:711.
Gasparini, F. J., R. B. Hochberg, and S. Lieberman. 1976. Biosynthesis of steroid sulfates by the boar testes. Biochemistry 15:3969-3975.
Gower, D. B. 1972. 16-Unsaturated C 19 steroids. A review of their chemistry, biochemistry and possible physiological role. J. Steroid Biochem. 3:45-103.
Gower, D. B. and R. L. Patterson. 1970. Identification of C19-16-unsaturated steroids in boar urine and spermatic vein plasma by combined gas chromatography and mass spectrometry. J. Endocrinol. 47:365-368.
Griffiths, N. M. and R. L. Patterson. 1970. Human olfactory responses to 5-alpha-androst-16-en-3-one—principal component of boar taint. J. Sci Food Agric. 21:4-6.
Groth, W. and R. Claus. 1977. [Relationships between concentrations of testosterone and the boar taint steroid 5alpha-androst-16-en-3-one in blood and adipose tissue; histometric finding in the testes of swine]. Zentralbl. Veterinarmed. A 24:103-121.
Hobkirk, R. 1985. Steroid sulfotransferases and steroid sulfate sulfatases: characteristics and biological roles. Can. J. Biochem. Cell Biol. 63:1127-1144.
Hobkirk, R., R. Renaud, and J. I. Raeside. 1989. Partial characterization of steroid sulfohydrolase and steroid sulfotransferase activities in purified porcine Leydig cells. J. Steroid Biochem. 32:387-392.
Igaz, P., E. Pap, A. Patocs, A. Falus, Z. Tulassay, and K. Racz. 2002. Genomics of steroid hormones: in silico analysis of nucleotide sequence variants (polymorphisms) of the enzymes involved in the biosynthesis and metabolism of steroid hormones. J. Steroid Biochem. Mol. Biol. 82:359-367.
Iida, A., A. Sekine, S. Saito, Y. Kitamura, T. Kitamoto, S. Osawa, C. Mishima, and Y. Nakamura. 2001. Catalog of 320 single nucleotide polymorphisms (SNPs) in 20 quinone oxidoreductase and sulfotransferase genes. J. Hum. Genet. 46:225-240.
Jakoby, W. B., R. D. Sckura, E. S. Lyon, C. J. Marcus, and J. L. Wang. 1980. Sulfotransferases. In: W. B. Jakoby (Ed.) Enzymatic Basis of Detoxification. pp. 1999-228. Academic Press, New York.
Kempster, A. J. and D. B. Lowe. Meat production with entire males. Growth performance and carcass characteristics as influenced by genotype and environment. 1993. Aarhus. 44th Annual Meating of the European Association of Animal Production.
Khalil, M. W. and V. Lawson. 1983. Steroids in porcine follicular fluid: analysis by HPLC, capillary GC and capillary GC/MS after purification on SEP-PAK C18 and ion exchange chromatography. Steroids 41:549-566.
Khalil, M. W., B. Strutt, D. Vachon, and D. W. Killinger. 1993. Metabolism of dehydroepiandrosterone by cultured human adipose stromal cells: identification of 7 alpha-hydroxydehydroepiandrosterone as a major metabolite using high performance liquid chromatography and mass spectrometry. J. Steroid Biochem. Mol. Biol. 46:585-595.
Kliewer, S. A., J. T. Moore, L. Wade, J. L. Staudinger, M. A. Watson, S. A. Jones, D. D. McKee, B. B. Oliver, T. M. Willson, R. H. Zetterstrom, T. Perlmann, and J. M. Lehmann. 1998. An orphan nuclear receptor activated by pregnanes defines a novel steroid signaling pathway. Cell 92:73-82.
Kouretas, D., S. Voyatzi, D. Sahpazidou, K. Lazopoulos, and O. Antonoglou. 1996. Enzymatic conversion of dihydrotestosterone from 3-keto to 3-enol form in the rat prostate. Anticancer Res. 16:2843-2848.
Kwan, T. K., D. B. Gower, D. J. Trafford, and Makin H.J. 1992. Gas chromatographic/mass spectrometric characteristics of 16-androstenes, saturated analogues and their derivatives. Biological Mass Spectrometry 21:160-164.
LaCroix, A. Z., K. Yano, and D. M. Reed. 1992. Dehydroepiandrosterone sulfate, incidence of myocardial infarction, and extent of atherosclerosis in men. Circulation 86:1529-1535.
Lin, Z., Y. Lou, and J. E. Squires. 2004. Molecular cloning and functional analysis of porcine SULT1A1 gene and its variant: a single mutation SULT1A1 causes a significant decrease in sulfation activity. Mamm. Genome 15:218-226.
Livak, K. J. and T. D. Schmittgen. 2001. Analysis of relative gene expression data using real-time quantitative PCR and the 2(-Delta Delta C(T)) Method. Methods 25:402-408.
Longcope, C. 1996. Dehydroepiandrosterone metabolism. J. Endocrinol. 150 Suppl:S125-S127.
Louveau, I., M. Bonneau, and D. B. Gower. 1991. Biosynthesis of 16-androstene steroids and testosterone by porcine testis tissue in vitro: effect of age and relationships with fat 5 alpha-androstenone levels in vivo. Acta Endocrinol. (Copenh) 125:526-531.
Lundstrom, K., B. Malmfors, L. Hansson, L. Edqvist, and B. Gahne. 1978. 5a-androstenone and testosterone in boars. Swed. J. Agric. Res. 8:171-178.
Luu-The, V., I. Dufort, N. Paquet, G. Reirnitz, and F. Labrie. 1995. Structural characterization and expression of the human dehydroepiandrosterone sulfotransferase gene. DNA Cell Biol. 14:511-518.
Lyle, S., J. Stanczak, K. Ng, and N. B. Schwartz. 1994. Rat chondrosarcoma ATP sulftirylase and adenosine 5′-phosphosulfate kinase reside on a single bifunctional protein. Biochemistry 33:5920-5925.
Makishima, M., A. Y. Okamoto, J. J. Repa, H. Tu, R. M. Learned, A. Luk, M. V. Hull, K. D. Lustig, D. J. Mangelsdorf, and B. Shan. 1999. Identification of a nuclear receptor for bile acids. Science 284:1362-1365.
Malmfors, B. and 0. Andresen. 1975. Relationship between boar taint intensity and concentration of 5a-androst-16-en-3-one in boar peripheral plasma and backfat. Acta Agric. Scand. 25:92-96.
Malmfors, B. and K. Lundstrom. 1983. Consumer reactions to boar meat: a review. Livest. Prod. Sci. 10:187-193.
Matsui, M., M. Takahashi, and H. Homma. 1993. Inhibition of rat liver hydroxysteroid sulfotransferase activity by alkylamines. Biochem. Pharmacol. 46:465-470.
Meerman, J. H., H. M. Sterenborg, and G. J. Mulder. 1983. Use of pentachlorophenol as long-term inhibitor of sulfation of phenols and hydroxamic acids in the rat in vivo. Biochem. Pharmacol. 32:1587-1593.
Negishi, M., L. G. Pedersen, E. Petrotchenko, S. Shevtsov, A. Gorokhov, Y. Kakuta, and L. C. Pedersen. 2001. Structure and function of sulfotransferases. Arch. Biochem. Biophys. 390:149-157.
Okuda, H., H. Nojima, N. Watanabe, and T. Watabe. 1989. Sulphotransferase-mediated activation of the carcinogen 5-hydroxymethyl-chrysene. Species and sex differences in tissue distribution of the enzyme activity and a possible participation of hydroxysteroid sulphotransferases. Biochem. Pharmacol. 38:3003-3009.
Otterness, D. M., C. Her, S. Aksoy, S. Kimura, E. D. Wieben, and R. M. Weinshilboum. 1995a. Human dehydroepiandrosterone sulfotransferase gene: molecular cloning and structural characterization. DNA Cell Biol. 14:331-341.
Otterness, D. M., H. W. Mohrenweiser, B. F. Brandriff, and R. M. Weinshilboum. 1995b. Dehydroepiandrosterone sulfotransferase gene (STD): localization to human chromosome band 19q13.3. Cytogenet. Cell Genet. 70:45-47.
Patterson, R. L. S. 1968. 5a-androst-16-ene-3-one: compound responsible for taint in boar fat. J. Sci. Food. Agric. 19:31-41.
Payne, A. H. and R. B. Jaffe. 1970. Comparative roles of dehydroepiandrosterone sulfate and androstenediol sulfate as precursors of testicular androgens. Endocrinology 87:316-322.
Pedersen, L. C., E. V. Petrotchenko, and M. Negishi. 2000. Crystal structure of SULT2A3, human hydroxysteroid sulfotransferase. FEBS Lett. 475:61-64.
Quintanilla, R., O. Demeure, J. P. Bidanel, D. Milan, N. lannuccelli, Y. Amigues, J. Gruand, C. Renard, C. Chevalet, and M. Bonneau. 2003. Detection of quantitative trait loci for fat androstenone levels in pigs. J. Anim Sci 81:385-394.
Raeside, J. I., H. L. Christie, and R. L. Renaud. 1999a. Androgen and estrogen metabolism in the reproductive tract and accessory sex glands of the domestic boar (Sus scrofa). Biol. Reprod. 61:1242-1248.
Raeside, J. I., H. L. Christie, and R. L. Renaud. 1999b. Metabolism of oestrone and oestradiol-17beta to conjugated steroids by the accessory sex glands of the male pig. J. Endocrinol. 163:49-53.
Raeside, J. I. and G. A. Howells. 1971. The isolation and identification of androstenediol sulfate from spermatic vein blood and testes of the boar. Can. J. Biochem. 49:80-84.
Raeside, J. I. and R. L. Renaud. 1983. Estrogen and androgen production by purified Leydig cells of mature boars. Biol. Reprod. 28:727-733.
Raeside, J. I., R. L. Renaud, and H. L. Christie. 1997. Postnatal decline in gonadal secretion of dehydroepiandrosterone and 3 beta-hydroxyandrosta-5,7-dien-17-one in the newborn foal. J. Endocrinol. 155:277-282.
Raeside, J. I., R. L. Renaud, and R. M. Friendship. 1989. Aromatization of 19-norandrogens by porcine Leydig cells. J. Steroid Biochem. 32:729-735.
Robbins, P. L. F. 1956. Identification of the enzymatically active sulfate as adenosine-3′-phosphate-5′-phosphosulfate. Journal of the American Chemistry Society 78:2652-2657.
Roberts, K. D. and S. Lieberman. 1970. The Biochemistry of the 3b-Hydroxy-delta-5 steroid sulftes. In: S. Bernstein and S. Solomon (Eds.) Chemical and Biological Aspects of Steroid Conjugation. pp. 291-320. Springer-Verlag, New York.
Roy, A. B. 1970. Enzymological aspects of steroid conjugation. In: S. Bernstein and S. Solomon (Eds.) Chemical and Biological Aspects of Steroid Conjugation. pp. 74-130. Springer-Verlag, New York.
Saini, S. P., J. Sonoda, L. Xu, D. Toma, H. Uppal, Y. Mu, S. Ren, D. D. Moore, R. M. Evans, and W. Xie. 2004. A novel constitutive androstane receptor-mediated and CYP3A-independent pathway of bile acid detoxification. Mol. Pharmacol. 65:292-300.
Schwarzenberger, F., G. S. Toole, H. L. Christie, and J. I. Raeside. 1993. Plasma levels of several androgens and estrogens from birth to puberty in male domestic pigs. Acta Endocrinol. (Copenh) 128:173-177.
Sellier, P. and M. Bonneau. 1988. Genetic relationship between fat androstenone level in males and development of male and female reproductive tracts. J. Anim. Breed. Genet. 105:11-20.
Shibutani, S., P. M. Shaw, N. Suzuki, L. Dasaradhi, M. W. Duffel, and I. Terashima. 1998. Sulfation of alpha-hydroxytamoxifen catalyzed by human hydroxysteroid sulfotransferase results in tamoxifen-DNA adducts. Carcinogenesis 19:2007-2011.
Sinclair, P. A., E. J. Squires, and J. I. Raeside. 2001a. Early postnatal plasma concentrations of testicular steroid hormones, pubertal development, and carcass leanness as potential indicators of boar taint in market weight intact male pigs. J. Anim Sci 79:1868-1876.
Sinclair, P. A., E. J. Squires, J. I. Raeside, J. H. Britt, and V. G. Hedgpeth. 2001b. The effect of early postnatal treatment with a gonadotropin-releasing hormone agonist on the developmental profiles of testicular steroid hormones in the intact male pig. J. Anim Sci. 79:1003-1010.
Singer, S. S., A. Z. Ansel, N. Van Brunt, J. Torres, and E. G. Galaska. 1984. Enzymatic sulfation of steroids—XX. Effects of ten drugs on the hepatic glucocorticoid sulfotransferase activity of rats in vitro and in vivo. Biochem. Pharmacol. 33:3485-3490.
Song, C. S., I. Echchgadda, B. S. Baek, S. C. Ahn, T. Oh, A. K. Roy, and B. Chatterjee. 2001. Dehydroepiandrosterone sulfotransferase gene induction by bile acid activated farnesoid X receptor. J. Biol. Chem. 276:42549-42556.
Squires, E. J., O. Adeola, L. G. Young, and R. R. Hacker. 1993. The Role of Growth-Hormones, Beta-Adrenergic Agents and Intact Males in Pork Production—A Review. Canadian Journal of Animal Science 73:1-23.
Squires, E. J., H. Deng, and L. Wu. Taint testing of pigs from the Ontario Carcass Appraisal Program. 0292, 43-47. 1992. University of Guelph, ON, Canada, OAC Publication. Ontario Swine Research Review.
Squires, E. J. and Y. Lou. Levels of boar taint in purebred entire male pigs. 0842-9839, 20. 1995. University of Guelph, ON, Canada, OAC Publication. Ontario Swine Research Review.
Squires, E. J. and K. Lundstrom. 1997. Relationship between cytochrome P450IIE1 in liver and levels of skatole and its metabolites in intact male pigs. J. Anim Sci 75:2506-2511.
Stahl, F., D. Schnorr, C. Pilz, and G. Dorner. 1992. Dehydroepiandrosterone (DHEA) levels in patients with prostatic cancer, heart diseases and under surgery stress. Exp. Clin. Endocrinol. 99:68-70.
Strott S. A. 1996. Steroid Sulfotranferases. Endocrine Reviews 17:670-697.
Tan, H. S. and J. I. Raeside. 1980. Developmental patterns of plasma dehydroepiandrosterone sulfate and testosterone in male pigs. Animal Reproduction Science 3:73-81.
Tanaka, M., T. Matsumoto, S. Yanai, M. Domukai, D. Toki, T. Hayashi, S. Kiuchi, H. Yasue, H. Uenishi, E. Kobayashi, and T. Awata. 2003. Conservation of the syntenies between porcine chromosome 7 and human chromosomes 6, 14 and 15 demonstrated by radiation hybrid mapping and linkage analysis. Anim Genet. 34:255-263.
Thomae, B. A., B. W. Eckloff, R. R. Freimuth, E. D. Wieben, and R. M. Weinshilboum. 2002. Human sulfotransferase SULT2A1 pharmacogenetics: genotype-to-phenotype studies. Pharmacogenomics. J. 2:48-56.
Tsai, M. J. and B. W. O'Malley. 1994a. Molecular mechanisms of action of steroid/thyroid receptor superfamily members. Annu. Rev. Biochem. 63:451-486.
Walstra, P. and A. W. Vermeer. 1993. Aspects of micro and macro economics in the production of young boars. Aarhus, Denmark. 44th Annual Meating of the European Association of Animal Production. 2:324.
Weinshilboum, R. and I. Aksoy. 1994. Sulfation pharmacogenetics in humans. Chem. Biol. Interact. 92:233-246.
Willeke, H., R. Claus, G. C. Mueller, F. Pirchner, and H. Karg. 1987. Selection for high and low levels of 5α-androst-16-en-3-one in boars I: Direct and correlated response of endocrinological traits. J. Anim. Breed. Genet. 104:64-73.
Willeke, H. and F. Pirchner. 1989. Selection for high and low levels of 5α-androst-16-en-3-one in boars II: Correlations between growth traits and 5-androstenone. J. Anim. Breed. Genet. 106:312-317.
Wood, T. C., C. Her, I. Aksoy, D. M. Otterness, and R. M. Weinshilboum. 1996. Human dehydroepiandrosterone sulfotransferase pharmacogenetics: quantitative Western analysis and gene sequence polymorphisms. J. Steroid Biochem. Mol. Biol. 59:467-478.
Wysocki, C. J. and G. K. Beauchamp. 1984.The ability to smell androstenone is genetically determined. USA. Proc. Natl. Acad. Sci. 81:4899.
Xu, Z., T. C. Wood, A. A. Adjei, and R. M. Weinshilboum. 2001. Human 3′-phosphoadenosine 5′-phosphosulfate synthetase: radiochemical enzymatic assay, biochemical properties, and hepatic variation. Drug Metab Dispos. 29:172-178.
Xue, J., G. D. Dial, E. E. Holton, Z. Vickers, E. J. Squires, Y. Lou, D. Godbout, and N. Morel. 1996. Breed differences in boar taint: relationship between tissue levels boar taint compounds and sensory analysis of taint. J. Anim Sci. 74:2170-2177.
Yokoyama, M. T. and J. R. Carlson. 1979. Microbial metabolites of tryptophan in the intestinal tract with special reference to skatole. Am. J. Clin. Nutr. 32:173-178.

What is claimed is:

1. An isolated polynucleotide which encodes a sulfotransferase protein comprising:
(a) a polynucleotide of SEQ ID NO:1;
(b) a polynucleotide which encodes a polypeptide comprising an amino acid sequence as depicted in SEQ ID NO:2;
(c) a polynucleotide which encodes a sulfotransferase protein that hybridizes under conditions of high stringency to the sequence in (a); or
(d) a polynucleotide with at least about 90% sequence identity to the sequences in (a).

2. The isolated polynucleotide of claim 1 comprising:
(a) a nucleotide change of a cytosine (C) to a thymine (T) at nucleotide position 219;
(b) a polynucleotide which encodes a sulfotransferase protein that hybridizes under conditions of high stringency to the sequence in (a); or
(c) a polynucleotide with at least about 90% sequence identity to the sequence in (a).

3. A vector comprising the polynucleotide of claim 1 operatively linked to a regulatory sequence.

4. A recombinant host cell comprising the vector of claim 3.

5. A vector comprising the polynucleotide of claim 1 operatively linked to a regulatory sequence.

6. A recombinant host cell comprising the vector of claim 5.

7. An isolated polypeptide having sulfotransferase activity comprising:
(a) an amino acid sequence as depicted in SEQ ID NO:2;
(b) a conservatively modified variant of the sequence in (a); or
(c) a sequence with at least about 90% sequence identity to the sequence in (a).

8. The isolated polypeptide of claim 7 wherein said polypeptide is a SULT2A1 hydroxysteroid sulfotransferase.

9. An antibody, or an antibody fragment thereof, that specifically binds to the polypeptide of claim 7.

10. A method of determining the genetic capacity of a male pig to develop boar taint comprising:
obtaining a sample of genetic material from a pig; and
detecting the levels of SULT2A1 expression, wherein a higher than average level of SULT2A1 indicates that said pig has a reduced susceptibility to developing boar taint.

11. The method according to claim 10 wherein said sample is selected from the group consisting of: liver, blood and testes.

12. The method according to claim 10 wherein said SULT2A1 comprises a amino acid sequence as depicted in SEQ ID NO:2.

13. A method for reducing or preventing boar taint in a pig comprising:
administering to a pig in sufficient quantity and under appropriate conditions a substance that increases the activity of a SULT2A1 enzyme to induce activity of said enzyme.

14. A method according to claim 13 wherein a nucleotide sequence encoding SULT2A1 enzyme is introduced into a pig.

15. A method for reducing or preventing boar taint in a pig comprising:
administering to a pig in sufficient quantity and under appropriate conditions a substance that induces or increases the expression of a SULT2A1 polynucleotide sequence to induce or increase the expression of said nucleotide sequence.

16. A method according to claim 5 wherein said nucleotide sequence is SEQ ID NO:1.

17. A method for screening for a substance that enhances metabolism of 16-androstene steroids in a pig comprising:
reacting a substrate of SULT2A1 and SULT2A1, in the presence of a test substance, under conditions such that sulfotransferase is capable of converting the substrate into a reaction product;
assaying for reaction product, unreacted substrate or unreacted SULT2A1;
comparing to controls to determine if the test substance selectively enhances SULT2A1 activity and thereby is capable of enhancing metabolism of 16-androstene steroids in a pig.

18. A method for screening for a substance that enhance metabolism of 16-androstene steroids by enhancing transcription and/or translation of the nucleotide sequence encoding SULT2A1 comprising:
culturing a host cell comprising a nucleic acid molecule containing a nucleic acid sequence encoding SULT2A1 and the necessary elements for the transcription or translation of the nucleic acid sequence, and optionally a reporter gene, in the presence of a test substance; and
comparing the level of expression of SULT2A1, or the expression of the protein encoded by the reporter gene with a control cell transfected with a nucleic acid molecule in the absence of the test substance.

19. A method of genetically typing pigs to determine those with desired boar taint characteristics comprising:
obtaining a sample of genetic material from a pig; and
assaying for the presence of a sulfotransferase allele characterized by a polymorphism in a SULT2A1 gene, said polymorphism being one which characterizes a first allele and a second allele which differ in activity of the SULT2A1 enzyme.

20. The method of claim 19 wherein said step of assaying is selected from the group consisting of: restriction fragment length polymorphism (RFLP) analysis, minisequencing, MALD-TOF, SINE, heteroduplex analysis, one base extension methods, single strand conformational polymorphism (SSCP), denaturing gradient gel electrophoresis (DGGE) and temperature gradient gel electrophoresis (TGGE).
